(19) United States
(12) Patent Application Publication
(10) Pub. No.: US 2017/0016047 A1
(43) Pub. Date: Jan. 19, 2017

(54) ENRICHING A SAMPLE OF CIRCULATING CELL-FREE NUCLEIC ACIDS

(71) Applicants: Oxford Gene Technology (Operations) Ltd, Begbroke Oxfordshire (GB); UCL Business PLC, London Greater London (GB)

(72) Inventors: Edwin Southern, Begbroke Oxfordshire (GB); Douglas Hurd, Begbroke Oxfordshire (GB); Dietrich Lueerssen, Begbroke Oxfordshire (GB); James Reid, Begbroke Oxfordshire (GB); Lyn Chitty, London Greater London (GB)

(21) Appl. No.: 15/124,215

(22) PCT Filed: Mar. 9, 2015
(86) PCT No.: PCT/GB2015/050681

§ 371 Date: Sep. 7, 2016

(30) Foreign Application Priority Data
Mar. 7, 2014 (GB) 1404063.8

(51) Int. Cl. C12Q 1/68

(52) U.S. Cl. CPC C12Q 1/6806; C12Q 1/6855; C12Q 1/686

(57) ABSTRACT

A method of enriching a sample of circulating cell-free DNA for DNA derived from a foetus or from a tumour includes carrying out amplification of DNA such that amplification of longer DNA molecules is discriminated against. This results in preferential amplification of foetal or tumour DNA, which can then be analysed using, for example, array comparative genome hybridisation.

The present invention relates to enriching a sample of circulating cell-free nucleic acids for nucleic acids from a first source. In particular, the nucleic acid may be derived from foetal nucleic acids or from a tumour.

Analysis of nucleic acids is used in many fields; for example, in the identification of infectious agents and of transformed cancer cells and in detecting abnormalities in human chromosomes responsible for developmental disorders, particularly for prenatal diagnosis. Historically, such analysis has only been possible using invasive techniques, such as biopsy of suspected tumours, and techniques such as amniocentesis, which carries a relatively high risk of miscarriage.

The discovery of circulating cell-free DNA in plasma has opened up the possibility of non-invasive techniques for the above-mentioned diagnoses. Circulating cell-free DNA is typically found in small amounts in a patient's bloodstream. It comprises sequences from a mixture of sources and is often composed of two sizes, a small sized fraction principally derived from apoptosis of cells and a larger sized fraction, thought to be derived from necrotic cells.

Circulating cell-free DNA has potential for diagnostics in at least two areas. Firstly, circulating cell-free DNA derived from tumour cells can be used as a ‘liquid biopsy’ to molecularly profile tumour DNA. Secondly, circulating cell-free DNA can be used to detect abnormalities in human chromosomes responsible for developmental disorders. There is growing interest in non-invasive detection of chromosome abnormalities in the unborn foetus, made possible by the observation that DNA from the foetus is present in the mother's bloodstream. A familiar example is trisomy of chromosome 21, associated with Down syndrome. Methods are available for the identification and analysis of such chromosomal abnormalities. The most widely used method for detection of deletions and amplification post-natally is microarray-based comparative genomic hybridisation (aCGH). In recent years, methods have been developed for detecting foetal chromosomal abnormalities by the analysis of circulating cell-free DNA from pregnant females using advanced sequencing methods.

Array CGH uses microarrays which comprise a solid surface which has been arrayed with a plethora of spots of individual DNAs designed to hybridise to different parts of the genome. Array CGH labels two DNA probes, the first is DNA from the sample of interest with the possible chromosomal aberrations, and the second is a reference DNA which is considered to be a normal DNA. The sample DNA is labelled with one fluorescent dye, the reference DNA is labelled with a spectrally distinct dye. The DNA probes are combined and then co-hybridised to a DNA microarray. After washing to remove unbound material the microarray is scanned to measure the amount of fluorescent dyes present on each of the individual spots of DNAs. A change of the ratio of the signal between the fluorescent signal of the sample and the reference is indicative of a chromosomal gain or loss. Conventionally the reference DNA used in aCGH may be DNA from another individual. However, this does not adequately allow for copy number variations, which may distort the measurements of the test sample leading to false results.

There are problems in analysing circulating cell-free DNA from plasma. First, the amount of circulating cell-free DNA in the bloodstream is much lower than that in cellular sources. Secondly, for prenatal diagnosis, at the time when intervention would be possible (i.e. in early stages of pregnancy) the amount of foetal DNA is small compared with the amount of the mother's own DNA. Equally, the amount of tumour DNA is small compared to the normal non-tumour DNA. Thirdly, the circulating cell-free DNA derived from the foetus is in small fragments, typical of fragments from apoptotic cells, whereas most of the maternal DNA is large. In a pregnant female, for example, foetal DNA generally falls within the size range of approximately 140-180 bp, with most of the maternal DNA being found in fragments that are around 1 kb or longer (Li et al. (2004) Clin. Chem. 50, 1002-11). There is also some suggestion that the smaller size of circulating cell-free DNA contains more tumour DNA in some cancers (e.g. prostate cancer) compared with the larger sized DNA fraction (Ellinger et al. (2008) Int. J. Cancer 122, 138-143).

As indicated above, the current state of the art, standard oligonucleotide array CGH, cannot be used to analyse circulating cell-free DNA for several reasons. Array CGH has a lower limit of approximately 200 ng of DNA, whilst the total amount of circulating cell-free DNA in a 5 ml blood sample is typically 25 ng. Amplification methods can be used with array CGH. However the current state of the art amplification methods for array CGH (for example, GenomePlex® DNA amplification, from Rubicon Genomics, sold by Sigma), if used for analysis of circulating cell-free DNA, favours amplification of longer background maternal/non-tumour DNA rather than the DNA of interest (e.g. tumour DNA or foetal DNA). Thus, amplification of the non-targeted fraction overwhelms the contribution of the targeted fraction, making the test less sensitive.

GenomePlex® amplification is a two-step method. The first step is the primer extension of the sample DNA using primers having a random sequence (to bind to the sample DNA) fused to a PCR primer binding site. Primer extension is carried out using a strand-invasive polymerase. The second step in the amplification consists of a PCR amplification using primers complementary to the primer binding site.

The GenomePlex® and similar amplification methods have been used for many years successfully in conjunction with array CGH. However, these methods do not successfully amplify the circulating cell-free DNAs of interest for non-invasive prenatal diagnosis or detection of tumour DNA. Therefore, in order to use a state-of-the-art amplification method, the background maternal DNA must be physically removed from the sample as far as possible. This might be done, for example, by separating the small fraction from the long fraction by gel electrophoresis, excising the band containing the short DNA, then purifying the DNA from the gel prior to carrying out amplification (Li et al. (2004)). EP 1 524 321 discloses a method in which the short fraction of DNA was physically separated from longer nucleic acid sequences in the sample prior to amplification. WO 2009/097511 discloses a method in which DNase-treatment of the DNA obtained from a maternal blood sample removes background maternal DNA prior to GenomePlex® amplification.

An alternative method for measuring the amount of a specific nucleic acid in a mixture of nucleic acids uses base sequence determination to analyse the composition of the sample. In one method, samples are amplified and sequenced using massively parallel shotgun sequencing (MPSS) of circulating cell-free DNA. In one example of this method, circulating cell-free DNA was directly sequenced from the plasma of pregnant women using high throughput shotgun sequencing technology. An average of five millions sequence tags per patient were obtained.

Each sequence tag (the short DNA fragment sequenced), also referred to as a “read” is mapped to its chromosome of origin, and the fragments per chromosome are enumerated. By counting the number of sequence tags mapped to each chromosome, any over or under-representation of chromosomes can be detected. In the case of Down syndrome, the percentage of chromosome 21 reads within the total amount of DNA reads in each plasma sample is calculated. As the foetal DNA content is usually between 4 and 20% of the total DNA in the plasma, any increment contributed by the foetus is diluted by the maternal contribution. This method does not require differentiation between the foetal and the maternal DNA (Fan et al. (2008), Proc. Natl. Acad. Sci. USA 105, 16266-71). However, this method requires batching of multiple samples, is expensive, slow and insensitive.

When used to analyse samples comprising small numbers of molecules, such as found in circulating cell-free DNA, it is standard practice to amplify the DNA by PCR after ligating adaptors to the ends of the fragments. In prior art next generation sequencing methods for detecting small amounts of DNA in a sample, it is standard practice to use annealing/extension times of around 60 to 90 seconds in order to maximise the amount of product obtained (Ehrich et al. (2011) Am. J. Obs. Gyn. 204, 205 e1-e11; Bianchi et al. (2012) Obs. Gyn. 119, 890-901; Fan et al. (2010) Clin. Chem. 56, 1279-86).

There are a number of other methods of DNA analysis which may be applied to measuring the level of a specific fraction in samples comprising a small amounts of nucleic acids.

WO 98/39474 discloses a method of non-invasive prenatal diagnosis that takes advantage of the presence of circulating foetal DNA within a pregnant female's bloodstream. In order to distinguish between maternal DNA and foetal DNA in the sample obtained from the bloodstream, the method relies upon analysing specific sequences within the foetal DNA that are inherited from the father (and thus are not present in the maternal DNA).

Many prior art methods involve amplifying specific pre-determined sequences in the DNA in the sample (see for example WO 03/062441, US 2011/0312503, US 2013/0178371, US 2013/0143213, and WO 2007/147063. These methods therefore require knowledge of the sequences to be amplified.

Other examples include analysis by the new sequencing methods (Chiu et al. (2011) Brit. Med. J., c7401), or testing methylation in differentially methylated DNA regions between mother and foetus using quantitative PCR to discriminate the methylated foetal DNA from the mother's non-methylated DNA (Papageorgiou et al. (2010) Nat. Med. 17, 510-13). However, these all require amplification as in all practical applications of these existing methods, the amount of cell-free nucleic acid circulating in the blood stream is too small to analyse without amplification. WO 2011/082386 also discloses a method that relies upon the difference in methylation levels of maternal DNA compared to foetal DNA. A methylation-sensitive enzyme digests hypomethylated foetal DNA, to which linkers containing a PCR primer-binding site may then be ligated. Linker-mediated PCR then preferentially amplifies unmethylated DNAs (i.e., those that were digested and include the linker). Reamplification is then required to provide sufficient product for array hybridisation.

The present invention seeks to provide an improved method of enriching a sample of circulating cell-free nucleic acids for nucleic acids from a first source.

According to an aspect of the present invention, there is provided a method of enriching a sample of circulating cell-free nucleic acids for nucleic acids derived from a first source, the sample comprising nucleic acids from the first source and nucleic acids from a second source, wherein the sample of cell-free nucleic acids includes nucleic acids falling within two size ranges, a shorter size range and a longer size range, wherein the nucleic acids from the first source are found in the shorter size range, the method including: a) forming templates for amplification from at least the nucleic acids in the shorter size range; and b) enriching the sample for the nucleic acids in the shorter size range by amplifying the templates to form amplification products in a manner independent of the nucleotide sequence of the nucleic acid having a sequence of interest.

This method allows differential amplification of nucleic acid fragments of different sizes. Specifically, the amplification discriminates against amplification of the longer fragments in a sample. The amplification of Step b) is carried out such that nucleic acids in the shorter size range are amplified more efficiently (and therefore preferentially) than nucleic acids falling within the longer size range (amplification of which is therefore discriminated against). As a result, the smaller fragments are effectively enriched, making it easier to detect and measure specific sequences within them against a background of longer fragments, whatever the means of analysis.

The sequence of interest is generally a sequence expected to be different in the condition being analysed (an aberrant region). The control sequence is generally a sequence in the test sample to which the amount of the sequence of interest is compared.

Ideally, substantially all of the nucleic acids from the first source will be amplified. Importantly, amplification is done non-specifically. The amplification is therefore not dependent upon the sequence of the nucleic acid having a sequence of interest. The efficiency of amplification is not dependent upon DNA sequence, but rather upon size of the initial template or of the template formed after the first round of amplification. It is therefore not necessary, with the present method, to have any prior knowledge of the specific nucleic acid sequences in the sample for the amplification step.

Whilst some background nucleic acids from the second source may also be amplified if any are present as small molecules, long nucleic acid fragments in the sample will generally not be amplified efficiently. The amplification step in the present method therefore enriches the sample for the small nucleic acids of interest whilst diluting the background nucleic acids originally present in large quantities in the sample. Advantageously, no step of physically separating the short nucleic acids from the long nucleic acids prior to amplification is necessary. In many embodiments both long and short nucleic acids are present in the sample at the start of the amplification of Step b). In some embodiments, at least some of the nucleic acids falling within the longer size range are present in the sample at the start of the amplification of Step b). With the present methods, the templates formed in Step a) from the short nucleic acid molecules are selectively amplified such that only nucleic acids falling within the shorter size range are amplified.

The templates for amplification and/or the amplification products have a size of less than approximately 750 bp or less than approximately 250 bp, for example. In a particular embodiment, the templates for amplification and/or the amplification products have a size of approximately 140-180 bp.

In some embodiments, the longest nucleic acids falling within the shorter size range are two to three times shorter than the shortest nucleic acids falling within the longer size range.

In a preferred embodiment, Step a) includes ligating adaptor molecules to the ends of the nucleic acids in the sample, the adaptor molecules including specific primer-binding sites. Step b) may include annealing primers complementary to the adaptor primer-binding sites, and amplifying the templates using polymerase chain reaction.

The use of adaptor PCR favours amplification of short fragments, and amplification is not dependent upon the specific sequence of the nucleic acids in the sample.

In many embodiments, the extension time used for annealing/extension during the amplification step is shortened compared to that which the skilled person would ordinarily seek to use. The combined annealing/extension time may be 30 secs or less, 20 secs or less, 10 secs or less, or may be as short as 5 secs or less. A shortened extension time is particularly useful where the percentage of nucleic acid derived from the first source is low (for example 4% or less). For example, in the case of detecting abnormality in foetal DNA, the foetal fraction can vary. This may be correlated, for example, to the mother's body mass index, to gestation age etc. The foetal fraction can vary from greater than 20% down to less than 4%. By reducing the extension time, there is decreased amplification of long DNA compared to the shorter DNA molecules. As a consequence, the detection limit of free foetal DNA is lowered, and a smaller (relative) amount of free foetal DNA can be detected.

The control sequence is preferably from a different chromosome to the nucleic acid sequence of interest.

In preferred embodiments, the sequence of interest is not suspected of being polymorphic and/or the control sequence is not suspected of being polymorphic.

According to another aspect of the present invention, there is provided a method of determining whether there is an increased or decreased amount of a nucleic acid having a sequence of interest derived from a first source in a sample of circulating cell-free nucleic acids, the sample comprising nucleic acids from the first source and nucleic acids from a second source, wherein the sample of cell-free nucleic acids includes nucleic acids falling within two size ranges, a shorter size range and a larger size range, wherein the nucleic acids from the first source are found in the shorter size range, the method including: a) forming templates for amplification from at least the nucleic acids in the shorter size range; b) enriching the sample for the nucleic acids in the shorter size range by amplifying the templates to form amplification products; and c) determining the relative amount of the amplification product of the nucleic acid having a sequence of interest by comparing to the amount of a control amplification product derived from the sample, the control amplification product having a control sequence, the control sequence being different to the sequence of interest.

In preferred embodiments, Step c) includes comparing the amount of amplification product with amplification product obtained from a set of reference nucleic acids corresponding to nucleic acids from the second source, wherein the reference nucleic acids include a nucleic acid having a sequence corresponding to the nucleic acid sequence of interest; wherein the reference nucleic acids do not include an increased or decreased amount of the nucleic acid having the corresponding sequence. Preferably, a ratio of the amount of amplification product from the sequence of interest compared to the control sequence is obtained, and this is compared to an equivalent ratio obtained from the set of reference nucleic acids.

The reference nucleic acids generally are not obtained from the same sample, but from a sample that preferably does not contain any nucleic acids from the first source. By “corresponding sequence”, it is meant an orthologous sequence that may or may not be identical to the sequence of interest. In preferred embodiments the corresponding sequence in the reference nucleic acids would be capable of binding under stringent conditions to a probe consisting of the sequence of interest.

In a most preferred embodiment the sequence of interest is not polymorphic, and so the corresponding sequence would be expected to be identical to the sequence of interest.

In an embodiment, Step c) includes: i) hybridising the amplification products to a test probe substantially complementary to the nucleic acid sequence of interest, and to at least one control probe, the control probe being substantially complementary to the control sequence; ii) co-hybridising the reference nucleic acids the to the test probe and to the control probe; iii) comparing the amount of hybridisation of the amplification products with the amount of hybridisation of the reference nucleic acids to the test probe and to the control probe to obtain a sample/reference test hybridisation ratio and a sample/reference control hybridisation ratio; and iv) comparing the test hybridisation ratio to the control hybridisation ratio to determine whether there was an increased or decreased amount of the nucleic acid having the nucleic acid sequence of interest in the first source.

The probes are preferably provided on a solid support, which may be a chip or a plurality of beads, for example. Where the solid support is a plurality of beads, each bead may include a single probe sequence. The solid support may have a porous surface.

In an embodiment, the amounts of the amplification products are compared using a method that does not involve microarray hybridisation. For example, the method may include determining the relative amount of the amplification product of the nucleic acid sequence of interest using a next generation sequencing method or a quantitative PCR method including digital PCR methods.

According to another aspect of the present invention, there is provided a method of predicting the likelihood of an abnormality in a subject, comprising determining whether there is an increased or decreased amount of a nucleic acid having a sequence of interest using a method as set out above, wherein if there is an increased or decreased amount of the nucleic acid having the sequence of interest, the subject is predicted to have an increased risk of an abnormality.

Step c) of the method may include comparing the amount of amplification product with amplification product obtained from a set of reference nucleic acids corresponding to nucleic acids from the second source, wherein the reference nucleic acids include a nucleic acid having a sequence corresponding to the nucleic acid sequence of interest; wherein the reference nucleic acids do not include an increased or decreased amount of the nucleic acid having the corresponding sequence.

The reference nucleic acids may have been obtained from the subject. Preferably, the reference nucleic acids have been obtained from cells from the subject that are not expected to contain nucleic acids from the first source. For example, the reference nucleic acids may have been obtained from the subject's white blood cells or from the subject's buccal cells.

In an embodiment, the reference nucleic acids have been extracted from nucleosomal DNA.

In one implementation of the method, the subject is a pregnant female and the abnormality is a foetus with a copy number defect, the nucleic acids from the first source are foetal nucleic acids and the nucleic acids from the second source are maternal nucleic acids.

The nucleic acid sequence of interest may be, for example, from chromosome 13, chromosome 18, chromosome 21 and/or the X chromosome. The control sequence may be from an autosome that is not chromosome 13, chromosome 18 or chromosome 21. The method may be a non-invasive method of prenatal diagnosis of Patau Syndrome, Edwards Syndrome, Down Syndrome or Turner Syndrome.

In another implementation of the method, the subject is suspected of having a tumour, wherein the first source is a tumour and the second source is non-tumour cells.

The control sequence is preferably selected so as not to include known benign copy number variations.

In an embodiment, the data obtained in Step c) are analysed using the Wilcoxon test to compare specific probe sets within a sample to controls derived from the same sample.

In an embodiment, a single p-value for a chromosome is obtained by combining p-values using the Stouffer method.

Preferred embodiments of the present invention enable the use of aCGH for the detection and analysis of minor components in a nucleic acid sample.

Preferred embodiments of the present invention are now described, by way of example only, with reference to the accompanying drawings, in which:

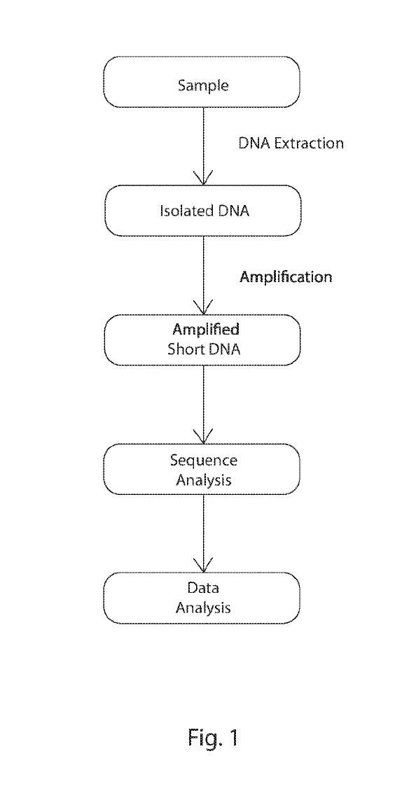

FIG. 1 is a flow-chart illustrating a general overview of a method of the invention and its use in detecting increase or decrease in the amount of a nucleic acid having a sequence of interest;

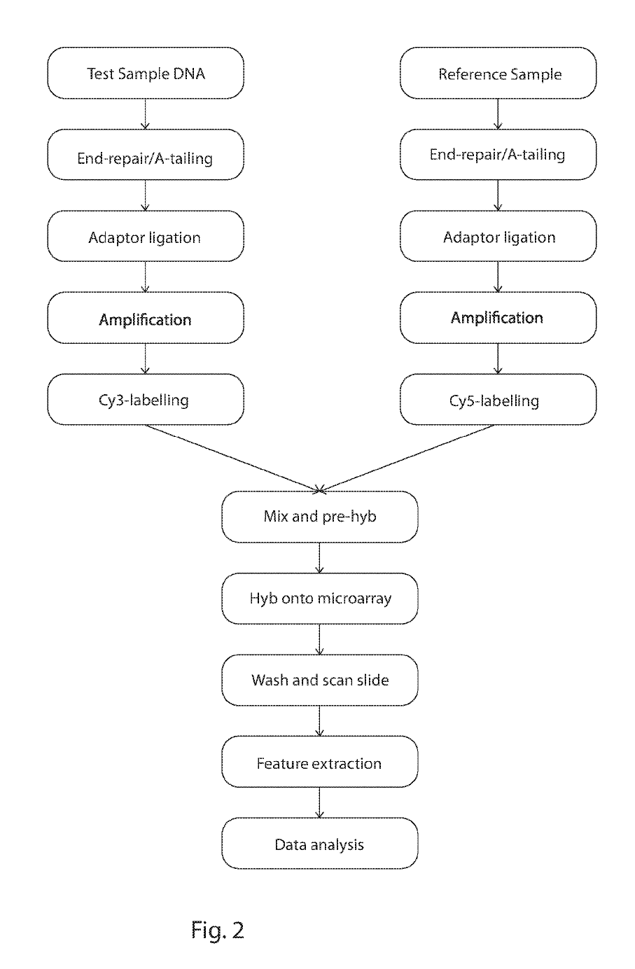

FIG. 2 is a flow-chart illustrating the steps of an embodiment of a method of the present invention and its use in detecting increase or decrease in the amount of a nucleic acid having a sequence of interest;

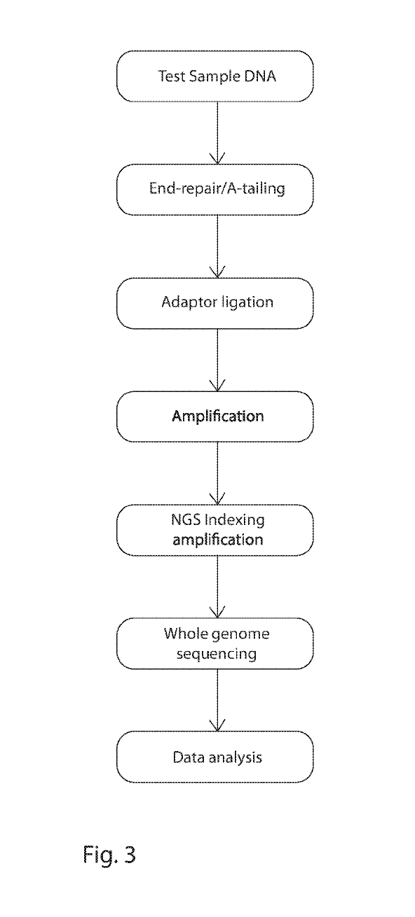

FIG. 3 is a flow-chart illustrating the steps of an embodiment of a method of the present invention and its use in detecting increase or decrease in the amount of a nucleic acid having a sequence of interest;

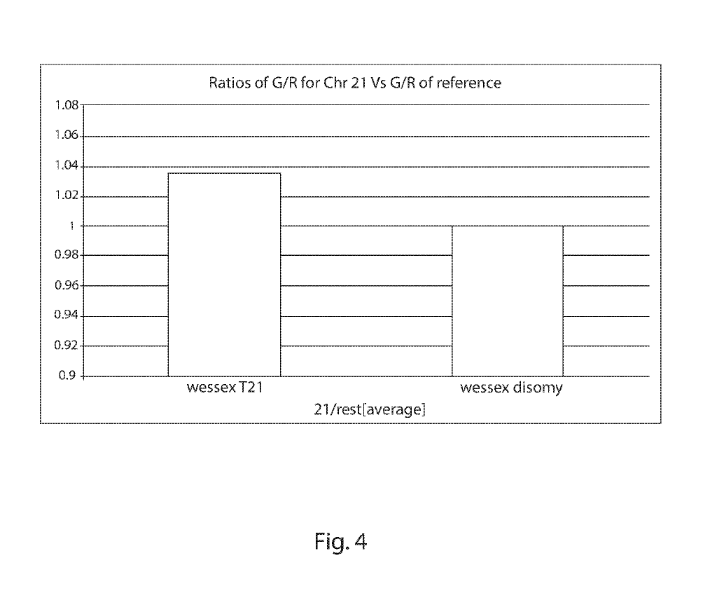

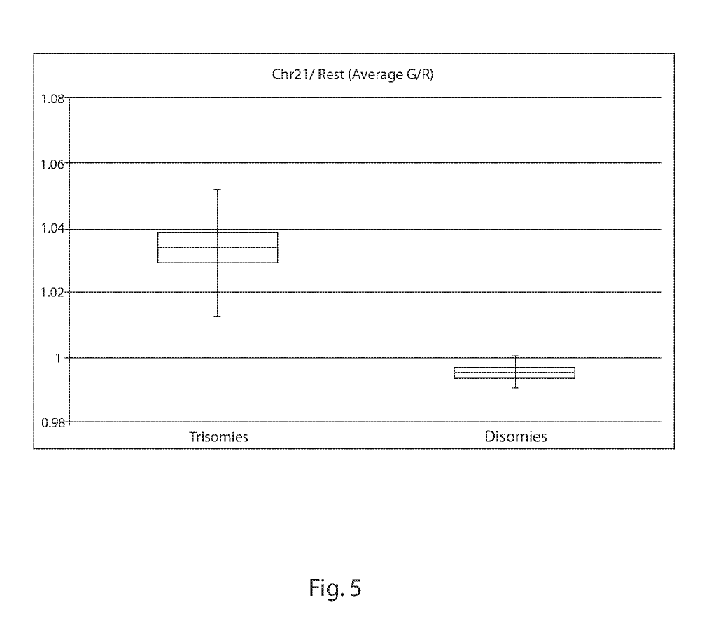

FIGS. 4 and 5 are bar graphs illustrating the results obtained in a method of using adaptor-PCR to detect trisomy 21;

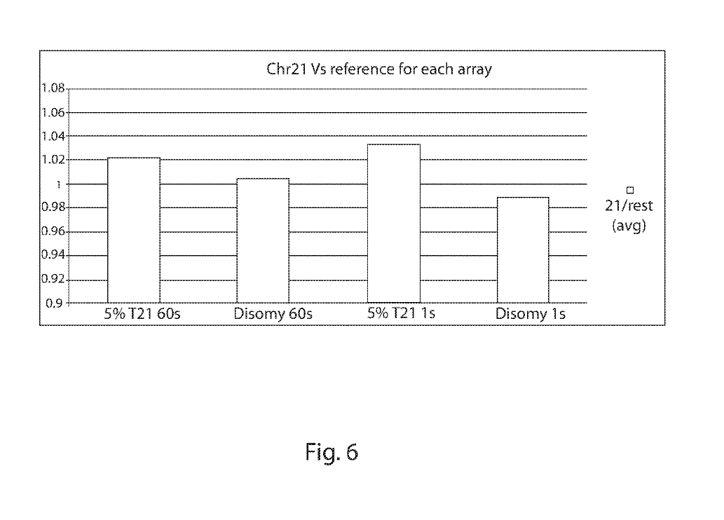

FIG. 6 is a bar graph comparing different extension times for detection of trisomy 21;

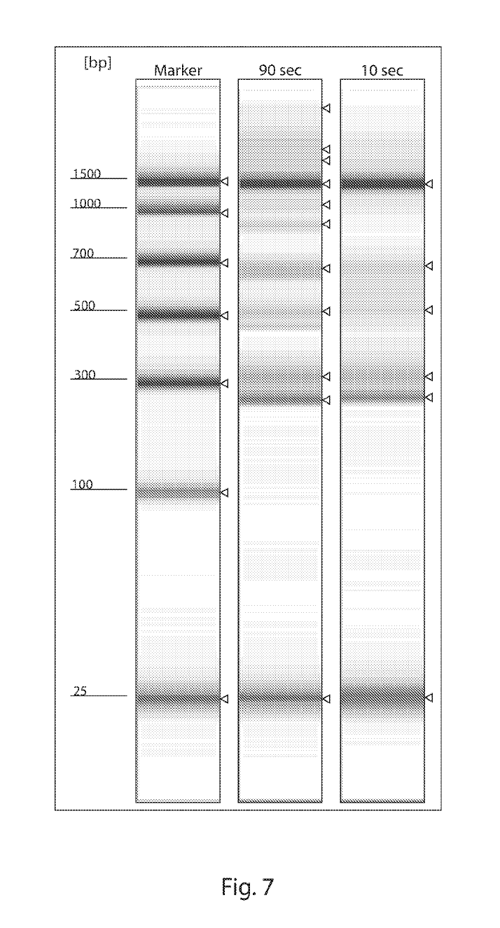

FIG. 7 is a photograph of DNA fragments separated by gel electrophoresis and demonstrating enrichment for shorter fragments;

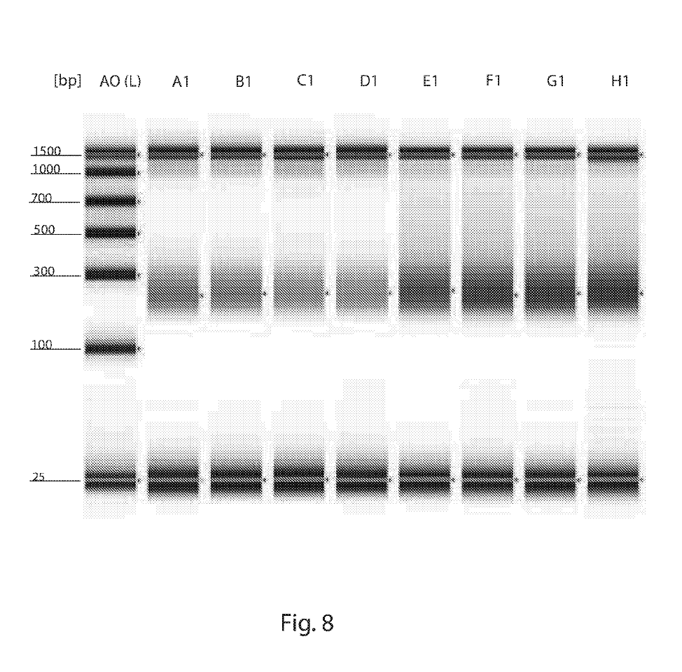

FIG. 8 is a gel image showing the yield of disomy DNA and trisomy 21 DNA products in adaptor PCR;

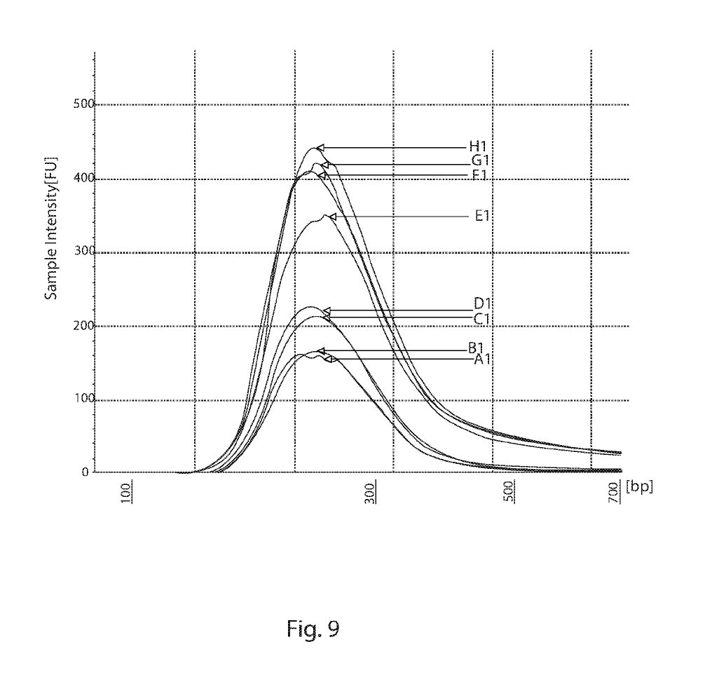

FIG. 9 is a graphical representation of the results in FIG. 8;

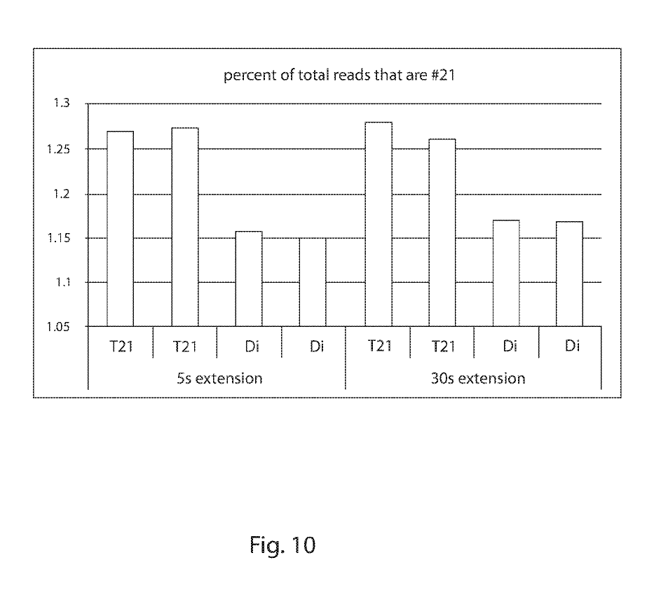

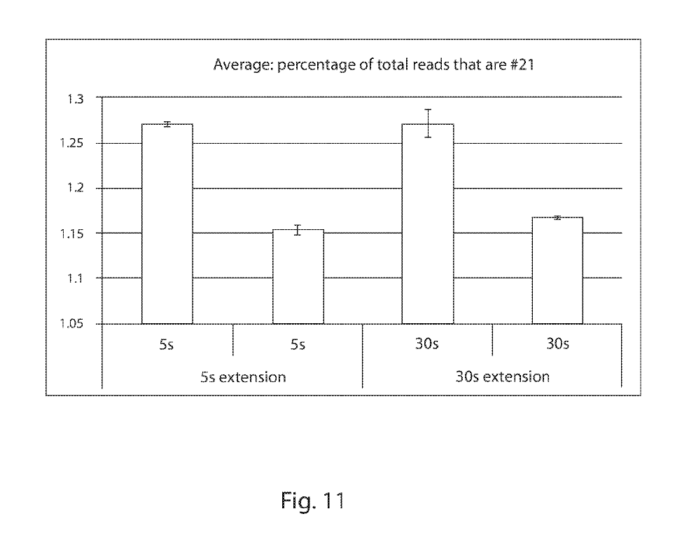

FIGS. 10 and 11 are bar graphs showing the percentage of total reads aligned to chromosome 21 for the disomy and trisomy samples with different extension times;

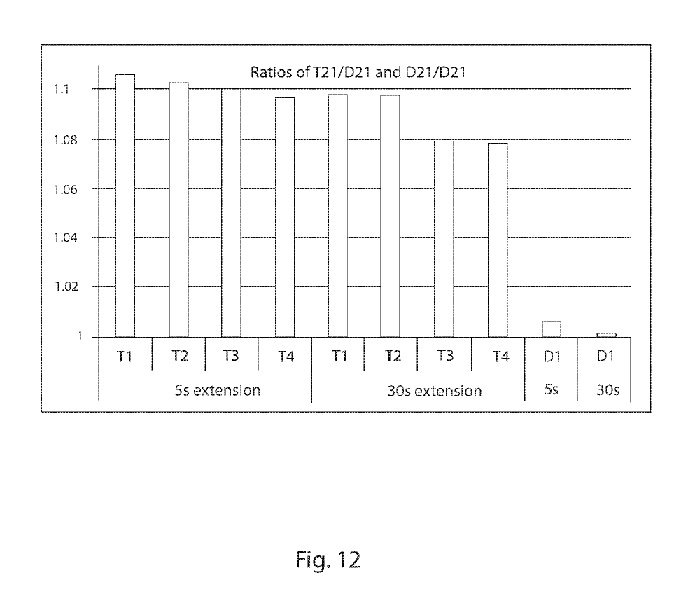

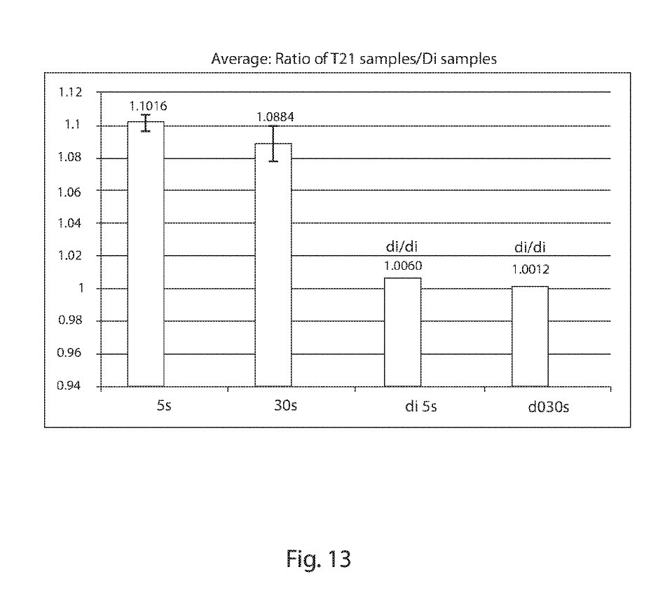

FIGS. 12 and 13 are bar graphs illustrating the ratio of ratios of trisomy/disomy samples with different extension times; and

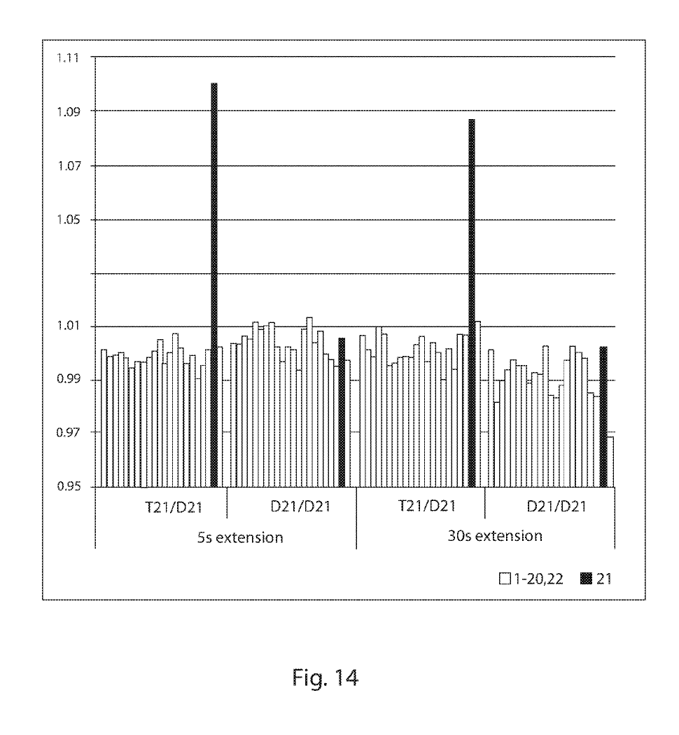

FIG. 14 is a bar graph illustrating the average ratios of trisomy/disomy samples and disomy/disomy samples across all of the autosomes.

FIG. 1 is a flow chart illustrating an overview of an exemplary method that may be used to enrich a sample of circulating cell-free DNA in accordance with a preferred embodiment of the invention. In a first step, circulating cell-free DNA is extracted from a sample taken from the bloodstream of the subject (for example, a pregnant female or a person suspected of having a tumour). The sample contains DNA of interest (for example, foetal or tumour DNA) and also background DNA (maternal or non-tumour DNA). DNA is extracted from the sample. The DNA is amplified, in accordance with the methods described below. The sample DNA may then be then analysed. This may be done in several ways. For example by co-hybridising to a microarray in aCGH (with a reference DNA corresponding to the background DNA), by using next generation sequencing methods, or using quantitative PCR. The amount of the amplification product derived from the sequence of interest in the sample may be compared to amplification product derived from a control sequence to ascertain whether or not there is an abnormal amount (more or less than should be expected) of a DNA sequence of interest.

The steps of an embodiment of the sample preparation method of the present invention are explained in detail below with particular reference to FIG. 2. FIG. 2 also sets out the steps of a possible method of analysing the prepared DNA using aCGH.

DNA Extraction

In a first embodiment of a method, the DNA sample is obtained from the bloodstream of a pregnant female, and the method may be a non-invasive method of prenatal diagnosis of trisomy 21. In another embodiment the sample may be from the bloodstream of a patient suspected of having prostate cancer, and the method may be a method of detecting tumour DNA. The method is equally applicable to detection of other congenital abnormalities caused by changes in copy number, such as trisomy 13 or trisomy 18, or to detection of other tumours. In any event, the DNA can be obtained simply from a blood sample from the subject in a straightforward manner without the need for any invasive sampling.

The DNA is extracted from the blood sample using standard methods well-known in the art (for example using the Qiagen QiAmp Circulating Nucleic Acid Kit). The DNA extracted from the sample includes a small amount of the DNA of interest (for example, foetal DNA or tumour DNA), which is present in the form of small fragments, alongside large amounts of background DNA (for example, maternal DNA or non-tumour DNA), the majority of which is in the form of longer fragments.

If the DNA is ultimately analysed using aCGH, reference DNA is also prepared. The reference sample used in currently known methods is often composed of a mixture of DNA from a number of individuals, in order that it represents a normalised population of genomic variation. The reference sample may be chosen to be a close match to the maternal DNA, preferably the pregnant female's own DNA.

There is a problem when one wants to measure the amount of a specific fraction within a test sample which comprises a mixture of sources. For example, the amount of DNA in the plasma of a blood sample from a pregnant woman is around 25 ng, and of this, typically 4-20% derives from the foetus. Thus, it may be desirable to use as a reference a DNA which is as close as possible in sequence to that of the pregnant mother. The reason for this is the presence in all humans of variation in the amounts of certain chromosomal regions, known as copy number variation. Similar concerns apply to the analysis of circulating tumour DNA. The presence of these variants in the reference will, of course, distort the measurements of the test sample, leading to false results. For the most accurate measurement of ratios (see below) it is important to match the size and quality of the reference DNA to the sample DNA.

It is also important that the size range of the DNA used as reference matches that deriving from the foetus or tumour. The foetal fraction appears to arise by apoptosis; it is around 150 bp with a narrow size range.

There are several potential sources of reference DNA for assaying circulating cell-free DNA.

The reference DNA for a method of prenatal diagnosis of trisomy 21 may be maternal DNA that is known not to comprise trisomy 21. For example, DNA from a (different) pregnant female carrying a non-trisomy 21 foetus could be used. Some, though not all, of the potential contribution of CNVs can be avoided by using the mother's DNA as reference, as the foetus has one of each chromosome pair from the mother and the other from the father. Preferably, the reference DNA includes DNA from the pregnant female in question, obtained in such a way that the chance of foetal DNA being present in the reference DNA is minimised. For example, the reference DNA could be obtained from maternal white blood cells. The reference DNA could be obtained from a buccal swab from the pregnant female. This DNA should not contain any foetal DNA, but would enable the presence of any copy number variations in the genome of the mother to be mitigated during the data analysis step. This therefore results in more accurate analysis of the foetal DNA.

Where the method is a method of identifying tumour DNA, the reference DNA may be DNA that is not suspected of containing tumour DNA. Again, this might be DNA obtained from a buccal swab from the subject.

It is possible to match the size range of the foetal DNA by extracting DNA from nucleosomes (preferably taken from the mother's circulating white cells, or from a source such as buccal cells as set out above). In a particularly preferred embodiment, for any of the above-mentioned sources of reference DNA, the reference DNA may be extracted from nucleosomal DNA (for example by extracting DNA from cells using the EZ Nucleosomal DNA Prep Kit (Zymo Research)). This should match closely the small DNA fraction as it is equivalent in size to DNA derived from apoptotic DNA.

Similarly, fragments suitable to use as a reference for circulating tumour DNA can be produced from the normal, untransformed cells of the patient taken from tissue sources and degrading it to fragments of the appropriate size range, by sonication or other means.

In the preferred embodiment, the reference DNA is extracted and treated in the same way as, and in parallel with, the sample DNA.

Amplification

As indicated above, the amounts of the DNA of interest present in the DNA sample are too small to be used directly in conventional aCGH.

In one embodiment, the DNA is amplified using an adaptor-PCR method, in which adaptors containing specific PCR primer-binding sites are ligated to the ends of the DNA fragments in the sample. The steps of this method are set out in FIG. 2. The adaptors are double stranded oligos of length and GC content that would allow them to include a PCR primer binding site of low sequence homology to any part of the human genome, and include a T-overhang to aid ligation to A-tailed double stranded genomic fragments. A preferred feature of the primers would be that their annealing temperature is the same as the optimal temperature of the polymerase, so that annealing and elongation may be carried out as a single step. In a preferred adaptor-PCR method, the fragments are subjected to gap repair, phosphorylation, adenylation, and then ligation of T-tailed adaptors in accordance with methods known by the skilled person.

PCR amplification is then preferably carried out with a shortened extension time. Surprisingly, the present applicant has determined that the extension time optimal for the disclosed methods is shorter than would be expected by the person skilled in the art. In a preferred method the annealing and extension would be carried out in a single step, at the same temperature. In a method of prenatal diagnosis, the preferred annealing and extension time should not exceed those needed to achieve the amplification of fragments greater than 150 bp for circulating foetal DNA. The exact length of the annealing/extension step depends on the amplification protocol being used, and the nature of the polymerase, for example. The person skilled in the art can readily determine how to optimise the extension conditions in order to achieve this goal. By way of example, for Pfu DNA-polymerase, under typical conditions, an annealing/extension time of 90 s is conventional. For the purposes of the present methods, however, the annealing/extension time may be approximately 30 sec or under. At a 30 sec combined annealing/extension time correct calling is achieved, and the applicant has even found that reducing the time below 30 sec improves the ratios and therefore the sensitivity of the detection due to fragments over approximately 700 bases not being amplified (see Examples 3 to 5 below).

The shortened extension time ensures that the extension reaction terminates before it reaches the end of long fragments. The primer-binding site at the end of the long fragments is thus not copied, and the copies of the long fragments are thus not available as template in the second amplification cycle. This amplification method therefore results in failure of long fragments in the sample to amplify, and resulting enrichment of the sample for the short fragments, which include the DNA of interest.

This approach addresses the problems of prior art amplification methods used to prepare samples for standard array oligonucleotide CGH in which large DNA fragments are readily amplified therefore diluting even further the DNA of interest found within the short fraction, and thus which cannot be used for pre-natal diagnosis or tumour identification. This amplification method allows detection of the low amounts of DNA in the foetal fraction when used with next generation sequencing methods (see below). The methods presented here avoid the requirement for a purification step in which the different size fractions are physically differentially separated prior to amplification, as the amplification methods discriminate against amplification of large fragments.

Typically a person skilled in aCGH and other methods of sequence analysis will seek to optimise conditions of amplification to give a large amount of product, enough to give good signals when analysed on a microarray. As can be seen, the preferred methods of amplification of the present application are designed to reduce the total amount of amplification product by avoiding amplification of longer fragments. This is counterintuitive to the person skilled in the art. As a consequence of the present amplification methods, signals on the microarray (see below) are lower. However, as this results from the smaller amount of the non-target signal, there is a lower background noise level and an improved measurement of the target signal.

The DNA prepared by the above method may then be analysed using aCGH as follows:

Labelling

Following or during amplification, the sample DNA may be labelled with fluorescent dye molecules that, following excitation, emit fluorescence at a specific wavelength.

The reference DNA is also labelled, but with an alternative dye emitting at a different wavelength to the first. The dyes may be Cy3 (green)/Cy5 (red) for example. The skilled person would appreciate that other suitable dyes could be used.

Standard array CGH labelling usually consists of primer extension using a strand-invasive polymerase such as exonuclease-free Klenow DNA polymerase incorporating labelled dNTP. This works well, although it may favour longer fragments over shorter fragments. An alternative labelling approach such as a chemical ligation approach, for example the ULS labelling method commercialised by Kreatech, which would have less of a size bias, could be used.

Labelling during the amplification could also be carried out, for example during the adaptor-PCR step the primers for the PCR could be fluorescently labelled. Alternatively, fluorescent dNTPs could be incorporated during the PCR by inclusion of fluorescently labelled dNTPs. Indirect labelling such as the inclusion of amino-allyl dNTP or biotin-dNTP in the PCR followed by a labelling using fluorescently labelled-NHS ester or fluorescently labelled-streptavidin respectively could alternatively be used.

There are many other suitable labelling methods available to the skilled person.

Co-Hybridisation to Microarray

The labelled, amplified sample and reference DNAs may be co-hybridised to a microarray including probes to a sequence of interest (a sequence suspected of being present in an increased or decreased amount relative to a normal source) and to control probes (complementary to nucleic acid sequences not expected to be present in an increased or decreased amount).

A nucleic acid array is a plurality of hybridisation probes immobilised on a solid surface to which target nucleotide sequences can be hybridised. This permits a sample to be contacted simultaneously with the plurality of probes in a single reaction compartment. The preparation and use of nucleic acid arrays are standard in the art.

Array CGH is a high-throughput method for analysing copy number variations at very low resolution levels across the genome. Variations in copy number are measured by hybridising both DNA test and reference samples to aCGH microarrays which contain locus-specific probes. For example, a microarray suitable for detecting trisomy of chromosome 21 in a foetus will include at least one probe (preferably at least 10, at least 100 or at least 1000 probes) specific to a sequence found on chromosome 21, and also at least one control probe (preferably at least 10, at least 100 or at least 1000 control probes) specific to sequences expected to be present in the foetus in the normal two copies. The probes will generally be in the range of 14 bases to 350 kb, for example 14 bases to 500 bases, 500 bases to 150 kb, or 150 kb to 350 kb. A preferred length for the probes might be 60 bases.

Commercially available microarrays (such as the CytoSure ISCA v2 (4×180 k) array by Oxford Gene Technology) can be used for the method disclosed herein of detecting congenital abnormalities, such as trisomy 21 in foetal DNA. This microarray contains probes specific to chromosome 21 and also probes specific to sequences that would not be suspected of being in anything other than two copies in the foetal DNA. The CytoSure ISCA v2 (4×180 k) array is in the form of a slide with four ˜154 k probe arrays on a single slide. These can be hybridised individually as four separate experiments. Most of the probes' target regions are spread relatively evenly across the genome (average probes' target region 25 kb apart) with a smaller number targeting ˜200 smaller regions of the genome that are of interest to cytogenetists with a higher density. The number of probe target regions on chromosome 21 is 1945; on chromosome 18 is 3628, and on chromosome 13 is 4922. For the remaining autosomal chromosomes there are 125026 probe target regions.

For the analysis of tumour DNA a probe to the nucleic acid sequence of interest would be derived from a chromosomal abnormality associated with cancer. Control probes would be derived from chromosomal regions not associated with cancer. A whole genome array such as the CytoSure ISCA v2 (4×180 k) array could be used. However if there are certain regions of the genome that are important to be analysed at greater resolution for the particular cancer, then the skilled person could design an array having increased probe density at these regions of interest.

Probes on microarrays can be designed with different optimisation parameters in mind. Comparison is not only between the green and red channels of a probe, but there is the need to compare this fluorescence ratio for a probe on one chromosome to a similarly performing probe on a different chromosome (see below). It is a point of discussion what constitutes a “similarly performing probe”. Among the factors that influence the performance are the melting temperature (roughly related to the GC content), the dye-bias (may change with signal intensity), and others. Since the aim is to measure regions of the genome that are present in ideally equal measures, the design can be carried out in order to avoid known regions with single nucleotide polymorphism, copy number variation, repeat regions, known break-points and translocation sites, highly variable regions and splice sites, for example. On the other hand, highly conserved regions are beneficial for probe selection.

Although commercially available arrays are suitable for use in analysing DNA prepared by the present methods, further improvements to reduce the noise level of microarray analysis can be made. For example, the number of probes targeting the regions of the genome that cause congenital abnormalities or transformation to cancer (as applicable) could be increased. In the case of congenital abnormalities this would favour an enhanced number of probes in chr 21 (Down syndrome), chr 13 (Patau syndrome) and/or chr 18 (Edwards syndrome). In other examples, there might also be enhanced probe coverage in chromosomes 4p (Wolf-Hirschhorn), 22q11.2 (diGeorge) and 5p (Cri du Chat). Many such syndromes are known. By enhancing the number of probes, the statistical power of the assay is increased and hence the confidence in detection of these syndromes in foetal DNA which is present in plasma DNA along with maternal DNA is enhanced. The present applicant has analysed the numerical relationship between the number of probes, the noise level in the data and the sensitivity of the test to give the number of probes preferably included on the array to detect a given level of the target DNA within the excess of other DNAs. For a specific experimental and analytical method used, the number of probes required to make a reliable call can be investigated. In one particular example, a number of approximately 2800 probes was sufficient for reliable calling of trisomy 21, while a random sample of the same probes (reduction by a factor of 5) produced insufficient accuracy, resulting in false negative calls.

Given that a non-parametric test (Wilcoxon) was used, a generalised model cannot be produced, but the skilled person can take advantage of the teachings herein to design suitable probe sets for a given application.

Eliminating probes with extreme base composition (for example, high GC content, or sequence repeats) and with poor hybridisation signals results in an enhanced confidence in detection of foetal or tumour DNA in a background of maternal or non-tumour DNA. This is because choosing optimal probes reduces the noise in the assay (often measured in aCGH by the DLRS metric) and improves the accuracy and precision of the ratio measurement (see below). Further improvements are achieved by matching the GC content of the probes in the target DNA region with those in the control DNA region, resulting in gains in the accuracy and precision of the ratios.

The design of the microarray could be improved compared to conventional aCGH designs by avoiding, rather than selecting, regions of known single nucleotide polymorphism and copy number variation. In other words, the probes used on the array preferably include sequences that would be expected to be identical in sequence (both test probes and control probes) and copy number (for control probes) in all individuals. This therefore enables stringent hybridisation conditions to be used so that only sequences exactly matching probes on the microarray are detected. This avoidance of single nucleotide polymorphisms and copy number variation leads to a signal ratio that is more predictably close to unity (see below). In conventional aCGH, by contrast, the detection of genomic regions where the signal ratio is different from unity is the goal, in order to detect chromosomal imbalances in a sample. The principle of avoiding single nucleotide polymorphism in aCGH is counter-intuitive for the purpose of detection of genomic abnormalities in a sample. Prior art methods (such as that disclosed in WO 98/39474) take advantage of differences in sequences inherited maternally and paternally by the foetus in order to distinguish between maternal and foetal DNA in the sample. The present approach to sample preparation preferably specifically avoids such genomic regions, and presents a novel, improved and non-obvious probe selection for the detection of larger-scale chromosomal imbalances in the foetus.

The labelled sample DNA and reference DNA are co-hybridised to the CGH microarray including probes for the DNA of interest and also a plurality of control probes. The microarray is then washed. The stringency of washing is determined by the level of mismatch that should be tolerated between the test and control probes and the sequences expected to be in the sample. As indicated above, ideally the probes are designed so as not to require any mismatch, such that stringent hybridisation and washing conditions should be used.

After washing, the microarray is scanned using commercially available apparatus such as, an Agilent microarray scanner. The amount of fluorescence on each individual spot is then calculated using specialist software (for example Agilent's feature extraction software).

After scanning and quantitation of the features on the array, the ratios between the sample DNA and the reference DNA are measured in the region of genomic DNA to be assayed (for example, chromosome 21 in Down syndrome) and compared to the ratio measurement in a control region (for example, chromosome 14). Any difference in the ratios is indicative of the region in the test DNA containing a copy number change.

In conventional aCGH, the fluorescence measurement on each probe is compared with that of the reference DNA to detect either an excess or a deficit in copy number. Ratios of fluorescence of sample:reference DNA for the test probes are thus obtained. An additional step may be carried out in which the test sample:reference ratio is compared with the equivalent ratio for chromosomal regions which are expected to be normal (i.e. control probes) to obtain a test:control ratio. For a normal sample, this ratio of ratios should be close to unity; any significant deviation from unity indicates deficit or excess.

Other solid surfaces could be used. The solid surface could be in the form of beads. The solid surface may be porous to increase its surface area.

Data Analysis

By labelling the test and reference samples with different colours and measuring signal intensities for these as described above, one can quantify the ploidy level in the DNA of the test sample compared to the reference sample. This quantification step involves taking the log 2 ratios of the sample to reference for each probe of the microarray so that the copy number variations are symmetric around 0. In an ideal situation, the log 2 ratio of normal (copy-neutral) is log 2(2/2)=0, single copy losses is log 2(1/2)=−1, and single copy gains is log 2(3/2)=0.58. In real applications, even after accounting for measurement error, the log 2 ratios may differ considerably from the theoretical values.

Following adequate normalisation of the aCGH data, which is mostly platform dependent (see for example Neuvial et al. (2006) BMC Bioinformatics 7, 264 for spatial normalisation), an initial step carried out is to smooth the data to possibly reduce the intricate technical noise of the system. Different smoothing methods have been proposed in the literature such as quantile smoothing and the use of wavelets (Eilers & de Menezes (2005) Bioinformatics 21, 1146-53; Hsu et al. (2005) Biostatistics 6, 211-26). The most important part of the analysis relies on the ability to reliably detect regions (segments of the genome) that exhibit aberrant copy numbers. The main aim is to identify the boundaries of regions that exhibit copy number changes with discrete levels of a sample compared to a reference in a noisy system. Segmentation of breakpoint detection methods detect copy number states of chromosomal sections and locate positions of transition between sections of different copy number states. The most widely used segmentation method is the Circular Binary Segmentation algorithm (Olshen et al. (2004) Biostatistics 5, 557-72). Once the segmentation results have been determined a call or probability of a copy number for each segment and importantly runs of segments is assigned.

A number of approaches have been devised to tackle this problem. For example, Hupé et al. (2004) (Bioinformatics 20, 3413-22) implement a Gaussian-based approach that models a piecewise constant function based on the Adaptive Weights Smoothing procedure. Hidden Markov models, where copy numbers are hidden states, extended this approach by taking into account their proximity on the genome as described in Fridlyand et al. (2004) (Journal of Multivariate Analysis 90, 132-53) and Marioni et al. (2006) (Bioinformatics 22, 1144-46).

The above data analysis method is known. The problem we address in the present invention is of an entirely different nature. Changes to the state-of-the-art data analysis are required, which are detailed here.

Here, a large portion of an entire chromosome (perhaps even an entire chromosome) may have a green-to-red ratio slightly above the value expected for a normal reference sample, but this value is not known a priori and is essentially a continuous variable that is related to the concentration of the excess DNA of interest. The change in the log 2 ratio can be on the order of log 2(1.05)=0.07, which is considerably smaller than the values typically considered in aCGH (compare with the discussion above). Hence, it is beneficial to combine results from a considerably larger number of probes to overcome experimental uncertainties that lead to noise in the signal with unspecified experimental origin. Robust estimates of the true value of the ratio based on the distribution of values may be combined with one another, such as the mean value, the median value, the trimmed mean, and M-estimators. In addition, statistical methods may be used to determine whether a sample belongs to a particular population (for example, in the case of detection of chromosomal aneuploidy of a foetal sample from maternal blood, whether or not the foetus is or is not affected by said aneuploidy). These statistical methods include, but are not limited to, two classes of methods: statistical classification and hypothesis testing. In statistical classification, the problem of membership to one or more categories is solved by evaluating measured properties for a number of known samples (the “training set”), and deriving a classification function for unknown samples. There are many known methods in the art, such as regression, cluster analysis, support vector machines, which by themselves represent examples of supervised and unsupervised learning methods. On the other hand, hypothesis testing evaluates the available data against an assumption (the null-hypothesis), and seeks to conclude whether the data is sufficiently different from the hypothesis that random variation cannot explain the difference between the observation and the expectation of the null-hypothesis. There are model-based hypothesis-testing methods such as the Student t-test or the Z-test, and non-parametric testing methods such as the Wilcoxon rank test, Mann-Whitney U-test and others. In addition to such frequentist hypothesis tests, Bayesian inference may be used. Depending on the specific problem to be solved, a person skilled in the art may select one method over another.

In an implementation, non-parametric testing using the Wilcoxon test was used for data analysis. It is important to point out that while the Wilcoxon test is part of analysis packages used for microarray experiments, its use is different from what is described below in the Examples. To be more specific, statistical testing using methods including the Wilcoxon test is used in the prior art to determine significant differences of specific probes or probe sets between samples where alternative samples act as controls, whereas in the present methods the significance testing is applied to testing specific probes sets within a sample against controls belonging to the same sample.

The sample preparation methods disclosed herein can be used to prepare nucleic acids for analysis using sequencing methods instead of aCGH. An outline of an exemplary method is illustrated in the flow chart of FIG. 3. When using a sequencing method, the adaptor sequences that are ligated on to the DNA fragment enable (or partially enable) the fragment to be sequenced by next generation sequencing techniques. An amplification step is carried out as described above, with a shortened extension time that preferentially amplifies the short DNA fragments. This amplification step may be used also to add additional DNA sequences that may be needed for the sequencing step. The amplification could take place directly on the flow cell of a sequencer, or if not on the flow cell, the DNA may be loaded on to a next generation sequencer. Following sequencing, the number of fragments of interest are compared to the number of DNA molecules from the control DNA.

Other analysis methods include qPCR, which may be used to determine the amount of DNA in the sample. If the region being tested is more methylated in the DNA of interest (foetal/tumour) DNA compared to the background DNA (maternal/non-tumour), then by using a shortened extension time in the amplification, there should be a differential amplification of the short fraction (more foetal/tumour DNA) compared to the long fraction (more maternal/non-tumour DNA), thereby resulting in the potential to increase the limit of detection of trisomy/tumours at a lower foetal/tumour fraction.

The above-described sample preparation method enables analysis of mixtures in which the targeted sequence is in small fragments within a background of larger non-targeted fragments, such as is found for the foetal fraction in the circulating cell-free DNA of a pregnant woman. With the present method, a step of shearing the DNA is unnecessary. The method takes advantage of shortened annealing/extension times for adaptor PCR, so that longer fragments are amplified less than shorter ones. That these amplification methods work is unexpected. The person skilled in the art of sequence analysis of samples with small numbers of molecules would expect shearing and long PCR cycles to give higher yields and more uniform coverage of the molecules from the sample.

### EXAMPLES

### Example 1

### Comparing GenomePlex® and Adaptor-PCR Amplification

Method

To illustrate the differences in amplification a model system was set up. Normal genomic DNA (Promega) was prepared which consisted of unsheared material (long) or sheared material (sheared to ˜180 base pairs using a Covaris Sonicator following the manufacturer's instructions and cleaned using Qiagen PCR purification columns). Trisomy 21 DNA (T21) from a cell line (Coriell) was sheared using a Covaris Sonicator.

The experiment was set up as follows:

Sample DNA:
1 ng sheared T21+25 ng Promega unsheared DNA
1 ng sheared T21+25 ng Promega sheared DNA
25 ng Promega DNA (negative control)

Reference DNA was prepared using sheared Promega DNA.

The sample and reference DNA were amplified using the GenomePlex amplification kit (Sigma) following the supplier's instructions. Briefly, 2 μl of library preparation solution was added, followed by 1 μl of library stabilisation solution. The DNAs were vortexed and placed on a thermal cycler at 95° C. for 2 mins. After incubation on ice, 1 μl of library preparation enzyme was added and the DNA was incubated under the following conditions: 15° C. for 20 mins, 24° C. for 20 mins, 37° C. for 20 mins, 75° C. for 5 mins. After incubation at 4° C., 15 μl of the DNA preparation was used for the next step. To the 15 μl of DNA, 7.5 μl of 10× amplification master-mix, 47.5 μl of water and 5 μl of WGA polymerase were added. The DNA was incubated at 95° C. for 3 mins and then cycled for 14 cycles at 94° C. for 15 seconds and 65° C. for 5 mins. The DNA was then cleaned up using Qiagen PCR purification columns using the manufacturer's instructions.

The amount of DNA was then quantitated using a nanodrop UV spectrophotometer following the manufacturer's instructions.

One microgram of the sample DNA was then labelled using Cy3 and 1 μg of reference DNA was labelled using Cy5 with the CytoSure labelling kit (Oxford Gene Technology). The DNA preparation was made up to 18 μl using water, and 10 μl of Random Primer and 10 μl of Reaction Buffer were added to make up a total volume of 38 μl. The DNA was denatured at 95° C. for 3 minutes. Following a 5 minute incubation on ice the following reagents were added and the DNA incubated for 2 hours: 10 μl of dCTP Labelling Mix, 1 μl of Cy-dCTP and 1 μl of exo free Klenow polymerase. After a 10 minute 65° C. step, the DNA was purified using a CytoSure purification column (Oxford Gene Technology) following the supplier's instructions. DNA was prepared for hybridisation to a 4×180 k CytoSure ISCA microarray (Oxford Gene Technology) by speedvac to dryness, resuspending the pellet in 40 μl of water, adding 5 μl of CotI (Kreatech), 11 μl blocking agent (Agilent) and 55 μl 2× hybridisation buffer (Agilent). The DNA was then hybridised to the array in a Surehyb chamber (Agilent) at 65° C. for 24 hours in a Surehyb oven rotating at 20 rpm (Agilent) following the supplier's instructions. Following hybridisation the microarray sandwich was disassembled under CGH buffer 1 (Agilent) and washed for 5 minutes at room temperature. The arrays were then washed for 1 minute at 37° C. with CGH Wash 2 (Agilent), then scanned in an Agilent microarray scanner at 2 μm resolution, 16 bit, 100% pmt. The TIF file was then feature-extracted using Agilent's feature extraction software and the data were analysed using Microsoft Excel. The Cy3 and Cy5 signals for each spot on the array were examined, with those with a signal intensity under 350 removed. The Cy3/Cy5 ratios calculated and the ratio of ratio calculated as follows.

$$Ratio of Ratio = \frac{Mean Cy 3 / Cy 5 ratio of probes on chr 21}{Mean Cy 3 / Cy 5 ratio of probes on chr 1 to 20}$$

In contrast an experiment was carried out using an alternative amplification method to the GenomePlex® random primer-PCR method.

To illustrate the differences in amplification a model system was set up. Normal genomic DNA (Promega) was prepared which consisted of unsheared material (long) or sheared material (sheared to ˜180 bp using a Covaris Sonicator following the manufacturer's instructions and cleaned using Qiagen PCR purification columns). Trisomy 21 DNA (T21) from a cell line (Coriell) was sheared using a Covaris Sonicator.

The experiment was set up as follows:

Sample DNA:
1 ng sheared T21+25 ng Promega unsheared DNA
1 ng sheared T21+25 ng Promega sheared DNA
25 ng sheared Promega DNA (negative control)

Reference DNA was prepared using sheared Promega DNA.

The sample and reference DNA were amplified using the Adaptor-PCR conditions (see below). The amount of DNA was then quantitated using a NanoDrop UV spectrophotometer following the manufacturer's instructions. The sample DNA and reference DNA were amplified using the OGT Next generation sequencing library preparation.

The sample (mixed size human DNA, Promega with 5% sheared T21 DNA, Coriell Cell Repositories) and reference DNA (sheared human DNA, Promega) were end-repaired and A-tailed in accordance with conventional protocols using a commercially available DNA polynucleotide kinase and DNA polymerases. DNA adaptors (for example, Agilent SureSelect XT Reagent Kit) were ligated to the end-repaired and A-tailed DNA by immediately adding 1.9 pmol of each adaptor hybrid and 12000 T4 Rapid DNA Ligase (Enzymatics) and incubating at 20° C. for 15 min. The reaction was purified using PCR Qiaquick columns and eluted in 30 μl Qiagen elution buffer. Half of the ligated DNA was then used in a PCR reaction with 1× Accuzyme reaction buffer (Bioline), 100 μmol each of the PCR primers targeting the adaptor sequences (e.g. Agilent SureSelect XT Reagent Kit), 50 nmol dNTPs (Bioline) and 5U Accuzyme DNA Polymerase (Bioline). Cycling conditions were as follows: 98° C. for 3 min followed by 15 cycles of 98° C. denaturation for 30 secs, 65° C. annealing for 30 secs and 72° C. extension for 1 min. The PCR reactions were cleaned-up using QiaQuick columns (Qiagen) and eluted in 30 μl Qiagen elution buffer.

The amount of DNA was then quantitated using a NanoDrop UV spectrophotometer following the manufacturer's instructions.

One microgram of the sample DNA was then labelled using Cy3 and 1 μg of reference DNA was labelled using Cy5 with the CytoSure labelling kit (Oxford Gene Technology). DNA was made up to 18 μl using water, and 10 μl of random primer and 10 μl of reaction buffer was added to make up a final volume of 38 μl. The DNA was denatured at 95° C. for 3 minutes. Following a 5 minute incubation on ice the 10 μl of dCTP Labelling Mix, 1 μl of Cy-dCTP and 1 μl of exo-free Klenow polymerase were added the DNA incubated for 2 hours. After a 10 minute 65° C. step, the DNA was purified using a CytoSure purification column (Oxford Gene Technology) following the supplier's instructions. DNA was prepared for hybridisation to a 4×180 k CytoSure ISCA microarray (Oxford Gene Technology) by speedvac to dryness, resuspending the pellet in 40 μl of water, adding 5 μl of CotI (Kreatech), 11 μl blocking agent (Agilent) and 55 μl 2× hybridisation buffer (Agilent). The DNA was then hybridised to the array in a Surehyb chamber (Agilent) for 65° C. for 24 hours in a Surehyb oven rotating at 20 rpm (Agilent) following the supplier's instructions. Following hybridisation the microarray sandwich was disassembled under CGH buffer 1 (Agilent) and washed for 5 minutes at room temperature. The arrays were then washed for 1 minute at 37° C. with CGH wash 2 (Agilent) then scanned in an Agilent microarray scanner at 2 μm resolution, 16 bit, 100% pmt. The TIF file was then feature-extracted using Agilent's feature extraction software and the data were analysed using Microsoft Excel. The Cy3 and Cy5 signals for each spot on the array were examined, with those with signal intensity under 350 removed. The Cy3/Cy5 ratios calculated and the ratio of ratio calculated as follows.

$$Ratio of Ratio = \frac{Mean Cy 3 / Cy 5 ratio of probes on chr 21}{Mean Cy 3 / Cy 5 ratio of probes on chr 1 to 20}$$

The results comparing the GenomePlex amplification and the adaptor-PCR are shown in Table 1 below:

TABLE 1

| | Amplification | | |
|---|---|---|---|
| | Method | Theoretical Ratio | Actual Ratio |
| 4% sheared T21 + | GenomePlex | 1.020 | 1.002 |
| 25 ng long genomic | | | |
| DNA | | | |
| 4% sheared T21 + | GenomePlex | 1.020 | 1.018 |
| 25 ng sheared | | | |
| genomic DNA | | | |
| No sheared T21 | GenomePlex | 1.000 | 0.998 |
| spiked + sheared | | | |
| genomic DNA | | | |
| (neg control) | | | |
| 4% sheared T21 + | Adaptor-PCR | 1.02 | 1.31 |
| 25 ng long genomic | | | |
| DNA | | | |
| 4% sheared T21 + | Adaptor-PCR | 1.02 | 1.19 |
| 25 ng Sheared | | | |
| genomic DNA | | | |
| Sheared genomic | Adaptor-PCR | 1.00 | 0.99 |
| DNA | | | |

The results show that when using the GenomePlex amplification the ratio of ratio obtained with the 4% sheared T21 spiked into the 25 ng sheared genomic DNA is close to the expected theoretical ratio of 1.020. By contrast the ratio of ratio for the 4% sheared T21 spiked into the 25 ng long genomic DNA was close to 1.000. This result indicates that the GenomePlex amplification method favours the amplification of the long genomic DNA. This demonstrates that known amplification methods for use with aCGH are not suitable where the DNA of interest is found within the short fraction of circulating cell-free DNA.

When using the Adaptor PCR amplification method the 4% sheared T21 DNA spiked into long genomic DNA gave a ratio significantly above the theoretical ratio. This shows that the adaptor-PCR amplification method favours amplification of short DNA and can be used to enrich a sample including mixed sizes of DNA for the short DNA fragments.

The applicant has shown that use of standard PCR methods such as GenomePlex do not work sufficiently well because signal from background long DNA (for example, maternal DNA) swamps the signal from the short DNA (for example, foetal DNA).

### Example 2

### Adaptor-PCR for Detection of Trisomy 21

Frozen cell-free plasma samples from pregnant women known to be carrying either disomy or T21 foetuses were provided by the Wessex National Genetics Reference Laboratory (NGRL). DNA extraction from 5 ml plasma per sample was carried out on one trisomy sample and three disomy samples using the QIAmp Circulating Nucleic Acid (Qiagen) kit following the manufacturer's instructions. DNA was eluted in 100 μl nuclease-free water, dried down using a SpeedVac and reconstituted with 15 μl nuclease-free water. The whole of each sample was end-repaired and A-tailed in accordance with conventional protocols. DNA adaptors (for example, Agilent SureSelectXT Reagent Kit) were ligated to the end-repaired and A-tailed DNA using a commercially available DNA ligase and following the manufacturer's instructions (e.g. T4 Rapid DNA Ligase, Enzymatics). The reaction was purified using QiaQuick columns (Qiagen) and eluted in 30 μl Qiagen elution buffer. Half of the ligated DNA was then used in a PCR reaction with 1× Accuzyme reaction buffer (Bioline), 50-200 μmol each of the PCR primers targeting the adaptor sequences (e.g. Agilent SureSelectXT Reagent Kit), 50 nmol dNTPs and 5U Accuzyme DNA Polymerase (Bioline). Cycling conditions were as follows: 98° C. for 3 min followed by 15 cycles of 98° C. denaturation for 30 secs, 65° C. annealing for 30 secs and 72° C. elongation for 1 min. The PCR reactions were cleaned-up using QiaQuick columns (Qiagen) and eluted in 30 μl Qiagen elution buffer.

The amount of DNA was then quantitated using a NanoDrop UV spectrophotometer following the manufacturer's instructions. One microgramme of the sample DNA was then labelled using Cy3 and 1 μg of reference DNA was labelled using Cy5 with the CytoSure labelling kit (Oxford Gene Technology). DNA was made up to 18 μl using water, 10 μl of Random Primer and 10 μl of Reaction Buffer was added. The DNA was denatured at 95° C. for 3 minutes. Following a 5 minute incubation on ice the 10 μl of dCTP Labelling Mix, 1 μl of Cy-dCTP and 1 μl of exo-free Klenow were added and the DNA incubated for 2 hours. After a 10 minute 65° C. step, the DNA was purified using a CytoSure purification column (Oxford Gene Technology) following the supplier's instructions. DNA was prepared for hybridisation to a 4×180 k CytoSure ISCA microarray (Oxford Gene Technology) by speedvac to dryness, resuspending the pellet in 40 μl of water, adding 5 μl of CotI (Kreatech), 11 μl blocking agent (Agilent) and 55 μl 2× hybridisation buffer (Agilent). The DNA was then hybridised to the array in a Surehyb chamber (Agilent) at 65° C. for 22 hours in a Surehyb oven rotating at 20 rpm (Agilent) following the supplier's instructions. Following hybridisation the microarray sandwich was disassembled under CGH buffer 1 (Agilent) and washed for 5 minutes at room temperature. The arrays were then washed for 1 minute at 37° C. in CGH buffer 2 (Agilent), and then scanned in an Agilent microarray scanner at 2 μm resolution, 16 bit, 100% PMT. The resulting TIF file was then feature-extracted using Agilent's feature extraction software and the data were analysed manually using Microsoft Excel.

To analyse the data, the Cy3 and Cy5 signals for each spot on the array were examined and all probes not designed to hybridise to human chromosomes and probes with green signals below 350 were removed. Green/red (Cy3/Cy5) ratios were calculated for every probe and the average green/red ratio was calculated for probes corresponding to chromosome 21, and probes corresponding to all other chromosomes except X and Y (reference probes). A ratio of ratios (Chr21/Reference) was then calculated for each sample set. The results are shown in FIG. 4, and demonstrate that the method can be used to detect an increased amount of chromosome 21 DNA compared to a disomy 21 control.

For all clinical samples tested to date, the results shown in FIG. 5 have been obtained. The boxes in the plot represent the range between the 1^{st} and 3^{rd} quartile of the ratios for each group, with the middle line representing the average value for all readings. The error bars indicate the maximum and minimum values obtained in each cohort. This demonstrates that this method of amplification following adaptor ligation makes possible the correct detection of trisomy and disomy 21 in foetuses non-invasively by using maternal plasma in the aforementioned samples.

### Example 3

### Adaptor-PCR with Shortened Extension Time

The sample (mixed size human DNA, Promega with 5% sheared T21 DNA, Coriell Cell Repositories) and reference DNA (sheared human DNA, Promega) were treated with end-repair, A-tailing and adaptor ligation as described above with respect to Example 2. The purified ligation products were used in PCR reactions as described previously, with different cycling parameters. Cycling conditions were as follows: 98° C. for 3 min followed by 15 cycles of 98° C. denaturation for 30 secs, 65° C. annealing for 30 secs and 72° C. extension. A 1 min extension time was tested against a 1 s extension time. The PCR reactions were cleaned-up using 1.8× AMPure XP magnetic beads (Beckman Coulter) and eluted in 30 μl nuclease-free water.

The amount of DNA was then quantitated using a NanoDrop UV spectrophotometer following the manufacturer's instructions. One microgramme of the sample DNA was then labelled using Cy3 and 1 μg of reference DNA was labelled using Cy5 with the CytoSure labelling kit (Oxford Gene Technology). DNA was made up to 18 μl using water, 10 μl of Random Primer and 10 μl of Reaction Buffer was added. The DNA was denatured at 95° C. for 3 minutes. Following a 5 minute incubation on ice the 10 μl of dCTP Labelling Mix, 1 μl of Cy-dCTP and 1 μl of exo free Klenow were added and the DNA incubated for 2 hours. After a 10 minute 65° C. step, the DNA was purified using a CytoSure purification column (Oxford Gene Technology) following the supplier's instructions. DNA was prepared for hybridisation to a 4×180 k CytoSure ISCA microarray (Oxford Gene Technology) by speedvac to dryness, resuspending the pellet in 40 μl of water, adding 5 μl of CotI (Kreatech), 11 μl blocking agent (Agilent) and 55 μl 2× hybridisation buffer (Agilent). The DNA was then hybridised to the array in a Surehyb chamber (Agilent) for 65° C. for 22 hours in a Surehyb oven rotating at 20 rpm (Agilent) following the supplier's instructions. Following hybridisation the microarray sandwich was disassembled under CGH buffer 1 (Agilent) and washed for 5 minutes at room temperature. The arrays were then washed for 1 minute at 37° C. in CGH buffer 2 (Agilent), and then scanned in an Agilent microarray scanner at 2 μm resolution, 16 bit, 100% PMT. The resulting TIF file was then feature-extracted using Agilent's feature extraction software and the data were analysed manually using Microsoft Excel.

To analyse the data, the Cy3 and Cy5 signals for each spot on the array were examined and all probes not designed to hybridise to human chromosomes and probes with green signals below 350 were removed. Green/red (Cy3/Cy5) ratios were calculated for every probe and the average green/red ratio was calculated for probes corresponding to chromosome 21, and probes corresponding to all other chromosomes except X and Y (reference probes). A ratio of ratios (Chr21/Reference) was then calculated for each sample set and the results shown Table 2 were obtained.

TABLE 2

| | 72° C. extension | |
|---|---|---|
| Sample | time | 21/ref |
| 5% T21 | 60 s | 1.022 |
| Disomy | 60 s | 1.004 |
| 5% T21 | 1 s | 1.033 |
| Disomy | 1 s | 0.988 |

These results are also illustrated in the bar graph of FIG. 6.

The results show a significant increase in the ratio for the trisomy samples from 60 secs to 1 sec extension time, and a significant decrease in the ratio for the disomies, leading to an increase in the difference between the trisomy and disomy ratios where a shorter extension time is used.

### Example 4

### Comparison of Long and Short Extension Times I

A sample of Hyperladder™ I (Bioline) was used and treated with end-repair, A-tailing and ligation as described above. PCR was carried out as described above, with different annealing/extension times and the samples were run on a D1K Tapestation (Agilent Technologies).

The results are shown in FIG. 7, which is gel image obtained using TapeStation Analysis Software (Agilent Technologies). Under standard conditions of amplification (90 sec annealing/extension: 65° C. for 30 sec, 72° C. for 60 sec), the fraction of the product in the size range <350 nt is approximately 36%. By contrast, under a shorter annealing/extension regime (10 sec annealing/extension: 65° C. for 10 sec), ˜61% are in this fraction. This is due to the bias against amplification of the longer fragments in the shorter annealing/extension regime. Specifically, in the longer annealing/extension 36% is >700 nt, as compared with none in the shorter annealing/extension.

### Example 5

### Comparison of Long and Short Extension Times II

The samples, mixed size disomy human DNA (Promega) and mixed size disomy human DNA (Promega) with 4% sheared T21 DNA (Coriell Cell Repositories), and reference DNA (sheared human DNA, Promega) were treated with end-repair, A-tailing and adaptor ligation as described above. The purified ligation products were used in PCR reactions as described previously, with different annealing/extension conditions. Cycling conditions were as follows: 98° C. for 3 min followed by 15 cycles of 98° C. denaturation for 30 secs, 65° C. annealing/extension. The annealing/extension times tested were 30 sec, 20 sec, 10 sec and 5 sec. The PCR reactions were cleaned-up using AMPure XP magnetic beads (Beckman Coulter) labelled and applied to arrays, and the signal data analysed as described above. The resulting ratios are shown in Table 3 below.

TABLE 3

| Annealing/extension | Chr21/Ref |
|---|---|
| time | Ratio |
| 30 s | 1.037 |
| 20 s | 1.046 |
| 10 s | 1.059 |
| 5 s | 1.066 |

This experiment shows that as the annealing/extension time decreases, the Chr21/Ref ratio significantly improves.

### Example 6

### Probe Optimisation

One objective during probe selection for the microarray can be that the factors to which the outcome of the comparison is known to be sensitive are kept constant during comparative probe selection. In practice, we have carried out hybridisation experiments to microarrays with an optimisation probe set. This optimisation probe set contained far more probes than necessary for the final design. The selection rules from this optimisation probe set were then chosen to maintain a similar distribution with respect to a certain variable (for example, GC content) across the chromosomes of interest (for example, chromosomes 13, 18 and 21 as well as a set of control chromosomes) while at the same time implementing constraints for average red/green signals for probes based on several optimisation experiments. For example, the GC content can be chosen to be stringently from a set window (for example, 20%<GC content<60%). Alternatively, multiple such windows can be set, and the proportion of probes within each window can be maintained over all chromosome sets. A similar approach can be taken with the signal intensities, such that either there is a set threshold for signals in the optimisation experiments, or that there are one or more windows of average, mean or median intensities for each probe (for example, 0-500, 500-1000, 1000-1500, etc.), among which the proportion of probes from different chromosomes or regions of interest is maintained. A person skilled in the art will recognise from these examples that probe sets can be grouped together based on performance criteria (for example, GC content, or signal level) to allow for a balanced design of the probes such that the probe sets of interest and the reference sets have similar numbers of probes within each such group, allowing analysis of balanced sets further downstream.

In contrast to the prior art, in the present methods we preferably look for regions that show differences between the sample and the reference on a probe-by-probe basis.

### Example 7

### Data Analysis

Simple data analysis may be carried out with the experimental method presented here. The data analysis starts from feature extracted microarray data, as is well-known in the art.

For a given sample, the following procedure has been used to find a difference between foetal trisomy and disomy samples from maternal blood samples:

(a) calculate, on a probe-by-probe basis, the green-to-red ratio for all probes;

(b) filter the data set by empirical criteria, such as by imposing a signal threshold appropriate to experimental conditions (for example, mean red and green signal in excess of 500 arbitrary fluorescent units (a.f.u));

(c) filter probes by their GC content (if this has not already been done during probe selection for the array);

(d) for the chromosome of interest (for example, chromosome 21), calculate the mean and median values of the filtered G/R ratios, and normalise them by a similarly obtained value for, alternatively, a fixed reference chromosome such as chromosome 14, or a set of appropriately chosen chromosomes (for example, all autosomes without significant risk of aneuploidy (i.e. preferably excluding chromosomes 13 and 18)).

These “ratios of ratios”, for the training set investigated, show a clear separation, which can be used to inform a classification threshold for a particular method. By way of example, the data shown in FIG. 5 may serve to illustrate this threshold, where the classification threshold between disomies (“normal” cases) and trisomies may be chosen to be around 1.03.

### Example 8

### Data Analysis Using the Wilcoxon Test

An alternative example of data analysis of the same data set is based on the Wilcoxon test. Here, the following procedure has been used:

(a) calculate, on a probe-by-probe basis, the green-to-red ratio for all probes;

(b) filter the data set by empirical criteria, such as by imposing a signal threshold appropriate to experimental conditions (e.g., mean red and green signal in excess of 500 arbitrary fluorescent units (a.f.u)); (c)

(c) filter probes by their GC content (if this has not already been done during probe selection for the array);

(d) for the reference

(e) use the value obtained in Step (d) to normalise the filtered green-to-red ratios for the chromosome of interest (for example, chromosome 21);

(f) carry out a Wilcoxon test for the population of values obtained in (e) against a null-hypothesis that the data is not different from the expected value for a foetus with disomy; theoretically, this value is 1; however, in certain situations the situation may arise where previous experiments carried out on training data (e.g., samples from disomy pregnancies) show that the expected value is different from 1; in that case it is permissible to make the required adjustments to the value of the null-hypothesis based on previous experience;

(g) choose a threshold for the p-value to determine the risk appropriate for a type-1 error (false positive). The precise value of such a threshold will depend on experimental details and will be chosen according to the performance requirements of the test. The person skilled in the art will be able to evaluate ROC curves to determine such thresholds. In an example, the threshold can be chosen to be 10⁻²⁰, which is substantially smaller than p-value cut-offs used in typical significance testing.

An example is given in Table 4 below, showing the ratios and p-values for patient samples.

TABLE 4

| | | c21/ | log2 | log2 | p-value |
|---|---|---|---|---|---|
| Sample | known status | control | (c21/c14) | (c21/control) | c21/control |
| A | disomy | 1.0050 | 0.0029 | 0.0072 | 2.16E−04 |
| B | disomy | 1.0137 | 0.0116 | 0.0196 | 5.25E−08 |
| C | disomy | 1.0148 | 0.0324 | 0.0212 | 1.51E−07 |
| D | disomy | 1.0209 | 0.0299 | 0.0299 | 7.19E−15 |
| E | disomy | 0.9903 | −0.0220 | −0.0141 | 8.93E−01 |
| F | disomy | 1.0114 | 0.0187 | 0.0163 | 9.81E−07 |
| G | trisomy | 1.0526 | 0.0602 | 0.0740 | 3.70E−59 |
| H | trisomy | 1.0487 | 0.0675 | 0.0686 | 1.91E−29 |

When the analysis method described above is used with a Student t-test as opposed to a Wilcoxon test in a preferred embodiment of the experimental method, the number of false positive and false negative may be higher compared to the preferred Wilcoxon test method.

### Example 9

### Comparing the Effect of Annealing/Extension Time on Trisomy 21/Disomy 21 Ratio

In this experiment a 10% spike-in (2.5 ng) of sheared either disomy DNA or T21 DNA (to model foetal DNA) was added to 25 ng size mix DNA (to model maternal DNA). The size mix consisted of a 1:1:1 weight ratio of high molecular weight: ˜1000 bp:180 bp. The samples were then prepared for NGS analysis by whole genome next generation sequencing using either a 30 sec or a 5 sec extension time in PCR1 and PCR2. It is proposed by the present applicant that the shorter extension time may favour amplification of the shorter sequences (or in the case of a real sample, the foetally-derived fragments) resulting in an increased T21/D21 ratio.

Methods

The amounts in the end repair/A-tailing reaction mix are given in Table 8 below.

TABLE 8

| End repair/A-tailing | Vol (μl) T21 | Vol (μl) Di |
|---|---|---|
| DNA size mix (10 ng/ul) | 2.5 | 2.5 |
| Spike in DNA (1 ng/ul) | 2.5 | 2.5 |
| Ligase Buffer | 10 | 10 |
| 8 mM dNTPs | 5 | 5 |
| 100 mM dATP | 0.5 | 0.5 |
| T4 Polymerase | 0.5 | 0.5 |
| T4 PNK | 1.1 | 1.1 |
| Klenow | 1.0 | 1.0 |
| TOP Polymerase | 3 | 3 |
| H₂O | 23.9 | 23.9 |
| Total | 50 | 50 |

One T21 and one disomy sample were set up and incubated at 20° C. for 30 min, and then 72° C. for 30 min. The samples were placed on ice. A ligation mix was set up by adding 2 μl Ligase Buffer to 4 μl Agilent adaptors (1:5) and 4 μl T4 Rapid Ligase. Five microlitres of ligation mix were added to each sample.

Ligations were incubated for 30 mins and then purified with 1.8× beads and eluted in 30 μl water. Samples were split into two and amplified for 10 cycles for either 30 or 5 secs with standard ramping rate on the Surecycler using the Agilent primers. (The number of cycles had been optimised so that no overamplification products were generated.) The cycling parameters were 98° C. for 3 mins followed by 10 cycles of 98° C. for 30 secs and 65° C. for either 30 or 5 secs.

A 5× mastermix of the Agilent SureSelect PCR1 reagents was made up of the following and 35 μl added to 15 μl library (see Table 9 below).

TABLE 9

| PCR1 | Vol (μl) |
|---|---|
| Library | 15 |
| Water | 25 |
| Primer 1 | 1.25 |
| (brown) | |
| Primer 2 | 1.25 |
| (white) | |
| Accuzyme | 5 |
| buffer | |
| 100 mM dNTPs | 0.5 |
| Accuzyme | 2 |
| Total | 50 |

Samples were purified using 1.8× ampure and quantified using the Qubit broad range DS DNA kit.

For PCR2, duplicates of 10 ng of PCR1 products were amplified for 6 cycles (8 and 10 cycles resulted in overamplification products) using the method above, incorporating indexed barcodes for multiplexed processing on the IIlumina MiSeq Desktop Next Generation Sequencer.

The pool for MiSeq sequencing is shown in Table 10 below.

TABLE 10

| | | | | Vol (μl) for | vol for | vol for |
|---|---|---|---|---|---|---|
| Sample | Index | ng/μl | nM | 10 μl of 4 nM | 200 μl | 500 μl |
| T21 5 s | 1 | 3.38 | 22.27 | 0.22 | 4.491 | 11.228 |
| T21 5 s | 2 | 5.75 | 37.88 | 0.13 | 2.640 | 6.600 |
| Di 5 s | 3 | 5.10 | 33.60 | 0.15 | 2.976 | 7.441 |
| Di 5 s | 4 | 7.38 | 48.62 | 0.10 | 2.057 | 5.142 |
| T21 30 s | 5 | 10.40 | 68.51 | 0.07 | 1.460 | 3.649 |
| T21 30 s | 6 | 13.50 | 88.93 | 0.06 | 1.124 | 2.811 |
| Di 30 s | 7 | 14.10 | 92.89 | 0.05 | 1.077 | 2.691 |
| Di 30 s | 8 | 17.00 | 111.99 | 0.04 | 0.893 | 2.232 |
| water | | | | 9.16 | 183.282 | 458.205 |

The final concentration of targets in the MiSeq pool was 4 nM so each sample was 4/8=0.5 nM. One hundred bases of both strands of each fragment were sequenced. Sequenced fragments or “reads”, were aligned to a chromosome in the human genome. Total reads for each chromosome were calculated. The number of reads aligned to chromosome 21 was calculated as a percentage of the total aligned reads.

Results

Table 11 shows the concentrations of the samples after PCR1 (Qubit broad range DS DNA kit).

TABLE 11

| | Concentration | Total |
|---|---|---|
| Sample | (ng/μl) | yield |
| B1. Trisomy 21/5 s | 3.68 | 118 ng |
| extension | | |
| C1. Disomy/5 s extension | 3.58 | 115 ng |
| D1. Trisomy 21/30 s | 9.18 | 294 ng |
| extension | | |
| E1. Disomy/30 s extension | 10.8 | 346 ng |

The DNA concentration as measured on the Qubit shows that the yield from the 30 sec extension is almost three times that of the 5 sec extension.

Table 12 and FIGS. 8 and 9 show the results of the samples after PCR2 (Qubit broad range DS DNA kit).

TABLE 12

| | Concentration | Total |
|---|---|---|
| Sample | (ng/ul) | yield |
| A1. Trisomy 21/5 s | 3.38 | 74.36 |
| extension | | |
| B1. Trisomy 21/5 s | 5.75 | 126.5 |
| extension | | |
| C1. Disomy/5 s extension | 5.1 | 112.2 |
| D1. Disomy/5 s extension | 7.38 | 162.36 |
| E1. Trisomy 21/30 s | 10.4 | 228.8 |
| extension | | |
| F1. Trisomy 21/30 s | 13.5 | 297 |
| extension | | |
| G1. Disomy/30 s extension | 14.1 | 310 |
| H1. Disomy/30 s extension | 17.1 | 376.2 |

These results are also shown in FIGS. 8 and 9, in which A0 is the Ladder, A1 is T21, 5 secs, Index 1, B1 is T21, 5 secs, Index 2, C1 is disomy, 5 secs, Index 3, D1 is disomy, 5 secs, Index 4, E1 is T21, 30 secs, Index 5, F1 T21, 30 secs, Index 6, G1 is disomy, 30 secs, Index 7 and H1 is disomy, 30 secs, Index 8.

Despite all samples having a starting amount of 10 ng, after PCR2 the 30 s extension again shows an almost three-fold greater yield than the 5 s extension.

There was no substantial difference in the peak target length of the extensions for 5 and 30 seconds. However, there was a substantial reduction in the high molecular weight products with the shorter extension time.

The sequencing data are summarised in Tables 13 and 14 below, with Table 13 showing the enumerated fragments aligned to each chromosome and Table 14 showing the percentages of total fragments aligned to each chromosome.

TABLE 13

| | 5 s | 5 s | 5 s | 5 s | 30 s | 30 s | 30 s | 30 s |
|---|---|---|---|---|---|---|---|---|
| | T21 | T21 | Di | Di | T21 | T21 | Di | Di |
| 1 | 288616 | 180132 | 146887 | 153806 | 206907 | 201796 | 186928 | 216592 |
| 2 | 294062 | 182600 | 149651 | 156763 | 213327 | 206431 | 191277 | 226019 |
| 3 | 233413 | 145817 | 119214 | 124517 | 170669 | 165432 | 154060 | 180609 |
| 4 | 223405 | 139423 | 113844 | 119094 | 166395 | 162931 | 149683 | 174727 |
| 5 | 212751 | 132468 | 108901 | 113148 | 156173 | 152031 | 140683 | 163627 |
| 6 | 203045 | 126659 | 104279 | 108628 | 148753 | 143995 | 135077 | 157437 |
| 7 | 190161 | 118687 | 97511 | 101465 | 138291 | 134811 | 125913 | 146728 |
| 8 | 175427 | 108863 | 89772 | 93263 | 126987 | 123417 | 114754 | 134741 |
| 9 | 144472 | 90270 | 73699 | 77315 | 104365 | 101331 | 94444 | 110368 |
| 10 | 172570 | 107576 | 87488 | 92276 | 123365 | 119828 | 111678 | 130594 |
| 11 | 160564 | 100391 | 81421 | 85397 | 115745 | 112184 | 104694 | 121161 |
| 12 | 155681 | 96466 | 79249 | 83177 | 113414 | 109704 | 101234 | 119342 |
| 13 | 111992 | 69891 | 56760 | 60043 | 83020 | 80743 | 74994 | 88477 |
| 14 | 106876 | 66950 | 54307 | 56577 | 78178 | 74975 | 69799 | 81979 |
| 15 | 100220 | 62442 | 51170 | 53065 | 72047 | 69473 | 65041 | 75643 |
| 16 | 107047 | 67305 | 54960 | 57562 | 74654 | 73021 | 69186 | 80090 |
| 17 | 97230 | 60710 | 49695 | 51802 | 67607 | 66751 | 61739 | 71629 |
| 18 | 90529 | 55624 | 46105 | 48487 | 65817 | 63665 | 59906 | 69680 |
| 19 | 69849 | 44378 | 35964 | 37900 | 48945 | 47426 | 43728 | 51520 |
| 20 | 73329 | 46401 | 37403 | 39526 | 51910 | 50391 | 46389 | 54705 |
| 21 | 49552 | 31026 | 23005 | 24041 | 35871 | 34495 | 29802 | 34539 |
| 22 | 43523 | 26826 | 21910 | 23101 | 29690 | 28822 | 26201 | 31388 |
| all chr | 3953534 | 2468167 | 2010703 | 2113776 | 2831445 | 2769597 | 2578023 | 2991421 |
| x | 186063 | 116570 | 96819 | 101269 | 134187 | 132338 | 123980 | 145059 |
| y | 2043 | 1283 | 1082 | 1110 | 1512 | 1372 | 1431 | 1539 |

The total number of aligned reads (excluding X and Y chromosomes) for each sample are shown in the italicised row. The range is between 2 and 4 million fragments or reads per sample. The total number of aligned reads in the sequencing lane is 21,716,666.

TABLE 14

| Data | 5 s | 5 s | 5 s | 5 s | 30 s | 30 s | 30 s | 30 s |
|---|---|---|---|---|---|---|---|---|
| analysis | T21 | T21 | Di | Di | T21 | T21 | Di | Di |
| 1 | 7.300 | 7.298 | 7.305 | 7.276 | 7.307 | 7.286 | 7.251 | 7.240 |
| 2 | 7.438 | 7.398 | 7.443 | 7.416 | 7.534 | 7.453 | 7.420 | 7.556 |
| 3 | 5.904 | 5.908 | 5.929 | 5.891 | 6.028 | 5.973 | 5.976 | 6.038 |
| 4 | 5.651 | 5.649 | 5.662 | 5.634 | 5.877 | 5.883 | 5.806 | 5.841 |
| 5 | 5.381 | 5.367 | 5.416 | 5.353 | 5.516 | 5.489 | 5.457 | 5.470 |
| 6 | 5.136 | 5.132 | 5.186 | 5.139 | 5.254 | 5.199 | 5.240 | 5.263 |
| 7 | 4.810 | 4.809 | 4.850 | 4.800 | 4.884 | 4.868 | 4.884 | 4.905 |
| 8 | 4.437 | 4.411 | 4.465 | 4.412 | 4.485 | 4.456 | 4.451 | 4.504 |
| 9 | 3.654 | 3.657 | 3.665 | 3.658 | 3.686 | 3.659 | 3.663 | 3.689 |
| 10 | 4.365 | 4.359 | 4.351 | 4.365 | 4.357 | 4.327 | 4.332 | 4.366 |
| 11 | 4.061 | 4.067 | 4.049 | 4.040 | 4.088 | 4.051 | 4.061 | 4.050 |
| 12 | 3.938 | 3.908 | 3.941 | 3.935 | 4.006 | 3.961 | 3.927 | 3.989 |
| 13 | 2.833 | 2.832 | 2.823 | 2.841 | 2.932 | 2.915 | 2.909 | 2.958 |
| 14 | 2.703 | 2.713 | 2.701 | 2.677 | 2.761 | 2.707 | 2.707 | 2.740 |
| 15 | 2.535 | 2.530 | 2.545 | 2.510 | 2.545 | 2.508 | 2.523 | 2.529 |
| 16 | 2.708 | 2.727 | 2.733 | 2.723 | 2.637 | 2.637 | 2.684 | 2.677 |
| 17 | 2.459 | 2.460 | 2.472 | 2.451 | 2.388 | 2.410 | 2.395 | 2.394 |
| 18 | 2.290 | 2.254 | 2.293 | 2.294 | 2.325 | 2.299 | 2.324 | 2.329 |
| 19 | 1.767 | 1.798 | 1.789 | 1.793 | 1.729 | 1.712 | 1.696 | 1.722 |
| 20 | 1.855 | 1.880 | 1.860 | 1.870 | 1.833 | 1.819 | 1.799 | 1.829 |
| 21 | 1.253 | 1.257 | 1.144 | 1.137 | 1.267 | 1.245 | 1.156 | 1.155 |
| 22 | 1.101 | 1.087 | 1.090 | 1.093 | 1.049 | 1.041 | 1.016 | 1.049 |

Chromosome 21 accounts for just over 1% of the total reads (as expected from the size of the chromosome and reported by others). It can be seen that the T21 spikes have a slightly higher value than the D21. This is shown graphically in FIGS. 10 and 11, which illustrate the percentage of total reads aligned to chromosome 21 for the disomy and trisomy samples with 5 s or 30 s extension times.

The left side of FIG. 10 represents the percentages of the 5 second extension time, and the right represents the 30 second extension time. The experiment was performed in duplicate and in FIG. 11 the individual samples are shown

FIG. 11 shows the average and the difference between the duplicates. It can be seen that both of the 10% T21 spike-ins have a higher percentage of aligned reads than the D21 spike-ins. FIG. 11 also indicates that the difference between disomy and trisomy is slightly greater for the 5 s extension than the 30 s. This is shown by calculating the ratios of the T21 reads/D21 reads (see Table 15 below, which shows the ratio of ratios of the trisomy/disomy samples with a 5 or 30 second extension step).

TABLE 15

| tri/di 5 s | tri/di 30 s | di/di 5 s | di/di 30 s |
|---|---|---|---|
| 1.0966 | 1.0971 | | |
| 1.1032 | 1.0984 | | |
| 1.0999 | 1.0783 | | |
| 1.1065 | 1.0797 | | |
| 1.1016 | 1.0884 | 1.0060 | 1.0012 |

As the disomy and trisomy samples were run in duplicate at each extension time, four ratios for the tri/di for each extension time could be generated and one di/di ratio. Table 15 shows that the average for the 5 s extension time is higher than the 30 s. One possible interpretation of this result is that the shorter extension time biases against extension of the longer fragments. The individual results and averages are shown graphically in FIGS. 12 and 13.

FIG. 14 compares the ratios of chromosome 21 to the other chromosomes, and illustrates the average ratios of trisomy/disomy samples and disomy/disomy samples across all of the autosomes.

FIG. 14 shows the average of four datapoints per chromosome for the trisomy/disomy ratios and a single disomy/disomy ratio datapoint, for all of the autosomes. The data for chromosome 21 are shown in the black bar. The data clearly show that the only ratios substantially different from a value of 1 are the trisomy/disomy ratios for chromosome 21 at both extension times (dotted line)

Conclusions

10% trisomy 21 spike in was detected by genome wide next generation sequencing using the described adaptor PCR amplification method with both a 5 and a 30 second extension time. Duplicate disomy and duplicate trisomy samples allowed for four trisomy/disomy ratios to be calculated. Although there is overlap, the average of the four T21/D21 ratios is 13% higher for the 5 s extension than the 30 s extension. One possible interpretation of this result is that the shorter extension time biases against amplification of the longer fragments. When all of the autosomes are analysed, the data shows that the only ratios substantially different from a value of 1 are the trisomy/disomy ratios for chromosome 21.

### Example 10

### Data Analysis Using the Stouffer Method

One hundred and seven samples (including controls) were collected, of which 24 were excluded due to apparent haemolysis of the samples. The test samples were blood samples from pregnant females with a high risk of having a trisomy 21 foetus (the controls were blood plasma from non-pregnant people). The DNA was then extracted from the samples using the QIAmp Circulating Nucleic Acid kit (Qiagen), and the DNA was amplified using the adaptor-PCR method as described above in Example 2. Following amplification, the DNA was labelled and together with labelled reference DNA (genomic DNA from a non-pregnant female) was hybridised to a microarray. The arrays were then washed and scanned, and the fluorescence at each spot was quantitated using feature extraction software as described in Example 2. The Cy3/Cy5 ratios were then calculated and analysed as follows.

For each sample, pairwise Wilcoxon rank sum tests were performed (Hollander & Wolfe (1999) Non Parametric Statistical Methods. New York: John Wiley & Sons) between the sample log-ratios grouped by chromosome location (with the exception of chromosome X) using Bonferroni multiple testing correction. The P-values obtained for chromosome 21 were then combined using the Stouffer method (Stouffer et al. (1949) The American Soldier, Volume 1: Adjustment During Army Life. Princeton University Press, Princeton). This results in a single p-value. Of the remaining 83 samples (including 11 trisomy 21 pregnancies), 65 were called as true-negatives, 9 were true-positives, and 9 were no cause due to high DLRs (high variation in the ratios). Typical cut-off values of the single p-value have been 0.01 in combination with sample or array quality scores.

The above Examples are provided to illustrate preferred embodiments of the invention. The skilled person will appreciate that modifications may be made thereto without departing from the scope of the claims.

In order to keep the length of the description of this patent application reasonable, an exhaustive description of all possible combinations of features has not been provided. All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another. All features described with respect to the pre-natal diagnosis methods of the subject matter of this application may be applied to the methods for identifying a tumour and vice versa. The skilled person will be capable of adapting the disclosure herein to apply to other tumours and foetal conditions not explicitly mentioned herein.

The disclosures in United Kingdom patent application 1404063.8, from which the present application claims priority, and in the abstract accompanying this application are incorporated herein by reference.

1. A method of enriching a sample of circulating cell-free nucleic acids for nucleic acids derived from a first source, the sample comprising nucleic acids from the first source and nucleic acids from a second source, wherein the sample of cell-free nucleic acids includes nucleic acids falling within two size ranges, a shorter size range and a longer size range, wherein the nucleic acids from the first source are found in the shorter size range, the method including:
a) forming templates for amplification from at least the nucleic acids in the shorter size range; and
b) enriching the sample for the nucleic acids in the shorter size range by amplifying the templates to form amplification products in a manner independent of a nucleotide sequence of a nucleic acid having a sequence of interest.

2. A method as claimed in claim 1, wherein the amplification products have a size of less than approximately 750 bp.

3. A method as claimed in claim 1, wherein the amplification products have a size of less than approximately 250 bp.

4. A method as claimed in claim 1, wherein the amplification products have a size of approximately 140-180 bp.

5. A method as claimed in claim 1, wherein a longest of the nucleic acids falling within the shorter size range are two to three times shorter than a shortest of the nucleic acids falling within the longer size range.

6. A method as claimed in claim 1, wherein Step a) includes ligating adaptor molecules to ends of the nucleic acids in the sample, each adaptor molecule including a primer-binding site.

7. A method as claimed in claim 6, wherein Step b) includes annealing primers complementary to the primer-binding sites, and amplifying the templates using polymerase chain reaction.

8. A method as claimed in claim 7, wherein the amplifying includes an annealing/extension step conducted for a time of no longer than 30 secs.

9. A method as claimed in claim 7, wherein the amplifying includes an annealing/extension step conducted for a time of less than 30 secs.

10. A method as claimed in claim 7, wherein the amplifying includes an annealing/extension step conducted for a time of approximately 20 sees or less than 20 secs.

11. A method as claimed in claim 7, wherein the amplifying includes an annealing/extension step conducted for a time of approximately 10 secs or less than 10 secs.

12. A method as claimed in claim 7, wherein the amplifying includes an annealing/extension step conducted for a time of approximately 5 secs or less than 5 secs.

13. A method as claimed in claim 1, wherein the control sequence is from a different chromosome to the nucleic acid sequence of interest.

14. A method as claimed in claim 1, wherein the sequence of interest is not suspected of being polymorphic.

15. A method as claimed in claim 1, wherein the control sequence is not suspected of being polymorphic.
